(19) United States
(12) Patent Application Publication
(10) Pub. No.: US 2026/0100258 A1
(43) Pub. Date: Apr. 9, 2026

(54) Medical Device With Radio Frequency Identification Component

(71) Applicant: Becton, Dickinson and Company, Franklin Lakes, NJ (US)

(72) Inventors: Ashley Rachel Rothenberg, Morris Plains, NJ (US); Kevin M. Ryan, Whitehouse Station, NJ (US)

(21) Appl. No.: 19/114,925

(22) PCT Filed: Sep. 25, 2023
(86) PCT No.: PCT/US2023/033605

§ 371 Date: Mar. 25, 2025

Related U.S. Application Data
(60) Provisional application No. 63/409,946, filed on Sep. 26, 2022.

(51) Int. Cl. G16H 10/65; G06K 19/07; G16H 20/13

(52) U.S. Cl. CPC G16H 10/65; G06K 19/0723; G16H 20/13

(57) ABSTRACT

A system may include a medical device, a radio frequency identification (RFID) tear tag attached to the medical device, wherein the RFID tear tag includes an RFID circuit and an electrical component electrically connected to the RFID circuit, wherein the electrical component comprises a first portion and a second portion, and wherein the first portion and the second portion are configured to be separable, wherein, when the electrical component is in a first state, the first portion and the second portion of the electrical component are electrically coupled and the RFID circuit is configured to provide a first signal, and wherein, when the electrical component is in a second state, the first portion and the second portion of the electrical component are not electrically coupled, and the RFID circuit is configured to provide a second signal.

### CROSS-REFERENCE TO RELATED APPLICATION

The present application claims priority to U.S. Provisional Patent Application No. 63/409,946, entitled “Medical Device with Radio Frequency Identification Component” filed Sep. 26, 2022, the entire disclosure of which is hereby incorporated by reference in its entirety.

### BACKGROUND

Radio frequency identification (RFID) may refer to a technology that uses electromagnetic fields to automatically identify and track devices, such as an RFID tag, that may be attached to an object. An RFID tag may consist of a radio transponder, a radio receiver, and a transmitter. When triggered by an electromagnetic interrogation pulse from a nearby RFID reader device, the RFID tag may transmit a signal that includes data, usually an inventory identification number, back to the RFID reader device. In some instances, the inventory identification number can be used to track goods.

RFID tags may include passive RFID tags and active RFID tags. A passive RFID tag may be powered by electromagnetic energy from an electromagnetic wave transmitted by an RFID reader device to interrogate an RFID tag. An active RFID tag may be powered by a power source included in the active RFID tag and, thus, can be read at a greater range by an RFID reader device, for example, up to hundreds of meters.

In some instances, RFID tags have been associated with medical devices to identify a medical device and/or a medicine included in the medical device. For example, an RFID tag may be included in packaging of a medical device, and the RFID tag may provide a signal that includes an identifier of a medical device and/or a medicine included in the medical device when interrogated by an RFID reader device.

However, an RFID tag may not provide accurate information with regard to the use of a medical device. For instance, a user, such as a doctor, may read the RFID tag on the packaging of a medical device at the time that the packaging is removed from the medical device. In such an instance, the doctor may not administer a medication from the medical device at the time that the packaging is removed and, therefore, the reading from the RFID tag on the packaging may not be accurate with regard to when the medical device was used. Further, the medical device may go unused after the packaging is removed.

### SUMMARY

Accordingly, provided are improved systems, devices, products, apparatus, and/or methods for determining a characteristic of a medical device using a radio frequency identification (RFID).

In one embodiment, a system includes a medical device, and a radio frequency identification (RFID) tear tag attached to the medical device, wherein the RFID tear tag includes an RFID circuit, and an electrical component electrically connected to the RFID circuit, wherein the electrical component comprises a first portion and a second portion, and wherein the first portion and the second portion are configured to be separable. When the electrical component is in a first state, the first portion and the second portion of the electrical component are electrically coupled and the RFID circuit is configured to provide a first signal. When the electrical component is in a second state, the first portion and the second portion of the electrical component are not electrically coupled and the RFID circuit is configured to provide a second signal.

In accordance with an embodiment of the present invention, when the first portion and the second portion of the electrical component are electrically coupled, the first portion and the second portion of the electrical component are conductively coupled.

In accordance with an embodiment of the present invention, the electrical component comprises an electrical conductor.

In accordance with an embodiment of the present invention, the electrical conductor comprises an electrically conductive trace.

In accordance with an embodiment of the present invention, the system further includes a base component, wherein the RFID circuit and the electrical component are positioned on the base component.

In accordance with an embodiment of the present invention, the base component includes a perforation, and wherein the first portion and the second portion are configured to be separable based on the perforation of the base component.

In accordance with an embodiment of the present invention, when the electrical component is in the first state, the first portion and the second portion are coupled together, and wherein when the electrical component is in the second state the first portion and the second portion cannot be recoupled.

In accordance with an embodiment of the present invention, the first portion and the second portion are configured to be separable independent of an additional mechanical component.

In accordance with an embodiment of the present invention, the electrical component is a first electrical component, and further includes a second electrical component electrically connected to the RFID circuit, wherein the second electrical component comprises an additional first portion and an additional second portion, and wherein the additional first portion of the second electrical component and the additional second portion of the second electrical component are configured to be separable. Wherein the additional first portion is separated from the additional second portion, the RFID circuit is configured to provide a third signal, wherein the third signal is different from the first signal and the second signal.

In accordance with an embodiment of the present invention, when the electrical component is in the first state, the RFID circuit is configured to provide the first signal during interrogation by an RFID reader device.

In accordance with an embodiment of the present invention, when the electrical component is in the second state, the RFID circuit is configured to provide the second signal during interrogation by an RFID reader device, wherein the second signal is different from the first signal.

In accordance with an embodiment of the present invention, the medical device includes a single-use medical device.

In accordance with an embodiment of the present invention, a medical device assembly includes a container having a closure, a radio frequency identification (RFID) circuit, and an electrical component electrically connected to the RFID circuit. The electrical component includes a first portion and a second portion, and wherein the first portion and the second portion are configured to be separable. The RFID circuit and the electrical component are positioned on the container, and when the electrical component is in a first state, the first portion and the second portion of the electrical component are electrically coupled and the RFID circuit is configured to provide a first signal during interrogation by an RFID reader device. When the electrical component is in a second state, the first portion and the second portion of the electrical component are not electrically coupled and the RFID circuit is configured to provide a second signal during interrogation by an RFID reader device, with the second signal being different from the first signal.

In accordance with an embodiment of the present invention, when the first portion and the second portion of the electrical component are electrically coupled, the first portion and the second portion of the electrical component are conductively coupled.

In accordance with an embodiment of the present invention, the electrical component comprises an electrical conductor.

In accordance with an embodiment of the present invention, the electrical conductor includes an electrically conductive trace.

In accordance with an embodiment of the present invention, the medical device further includes a base component, wherein the RFID circuit and the electrical component are positioned on the base component, and wherein the base component is positioned on the closure of the container.

In accordance with an embodiment of the present invention, the base component includes a perforation, and wherein the first portion and the second portion are configured to be separable based on the perforation of the base component.

In accordance with an embodiment of the present invention, the first portion and the second portion of the electrical component are configured to be destructively disconnected when the electrical component is in the second state.

In accordance with an embodiment of the present invention, the first portion and the second portion are configured to be separable independent of an additional mechanical component.

In accordance with an embodiment of the present invention, the electrical component is a first electrical component, and the medical device assembly further includes a second electrical component electrically connected to the RFID circuit. The second electrical component includes an additional first portion and an additional second portion, and wherein the additional first portion of the second electrical component and the additional second portion of the second electrical component are configured to be separable. The additional first portion is separated from the additional second portion, and the RFID circuit is configured to provide a third signal, wherein the third signal is different from the first signal and the second signal.

In accordance with an embodiment of the present invention, the container is a single-use container.

### BRIEF DESCRIPTION OF THE DRAWINGS

Additional advantages and details are explained in greater detail below with reference to the exemplary embodiments that are illustrated in the accompanying schematic figures, in which:

FIG. 1 is a diagram of a non-limiting aspect or embodiment of a system for determining a characteristic of a medical device using a radio frequency identification (RFID) tag;

FIG. 2 is a diagram of a non-limiting aspect or embodiment of components of one or more devices and/or one or more systems of FIG. 1;

FIG. 3 is a flowchart of a non-limiting aspect or embodiment of a process for determining a characteristic of a medical device using an RFID tag;

FIGS. 4A and 4B are diagrams of a non-limiting embodiment of an RFID tear tag;

FIG. 5 is a diagram of a non-limiting embodiment of an RFID tear tag;

FIG. 6 a diagram of a non-limiting embodiment of a medical device assembly including an RFID tear tag attached to a container;

FIG. 7 a diagram of a non-limiting embodiment of a medical device assembly including an RFID tear tag attached to a syringe;

FIG. 8 a diagram of a non-limiting embodiment of a medical device assembly including an RFID tear tag attached to an intravenous (IV) assembly;

FIG. 9 a diagram of a non-limiting embodiment of a medical device assembly including an RFID tear tag attached to medication packaging; and

FIG. 10 a diagram of a non-limiting embodiment of positions of RFID reader devices.

### DETAILED DESCRIPTION

It is to be understood that the present disclosure may assume various alternative variations and step sequences, except where expressly specified to the contrary. It is also to be understood that the specific devices and processes illustrated in the attached drawings, and described in the following specification, are simply exemplary and non-limiting embodiments. Hence, specific dimensions and other physical characteristics related to the embodiments disclosed herein are not to be considered as limiting.

For purposes of the description hereinafter, the terms “end,” “upper,” “lower,” “right,” “left,” “vertical,” “horizontal,” “top,” “bottom,” “lateral,” “longitudinal,” and derivatives thereof shall relate to embodiments or aspects as they are oriented in the drawing figures. However, it is to be understood that embodiments or aspects may assume various alternative variations and step sequences, except where expressly specified to the contrary. It is also to be understood that the specific devices and processes illustrated in the attached drawings, and described in the following specification, are simply non-limiting exemplary embodiments or aspects. Hence, specific dimensions and other physical characteristics related to the embodiments or aspects of the embodiments or aspects disclosed herein are not to be considered as limiting unless otherwise indicated.

No aspect, component, element, structure, act, step, function, instruction, and/or the like used herein should be construed as critical or essential unless explicitly described as such. Also, as used herein, the articles “a” and “an” are intended to include one or more items, and may be used interchangeably with “one or more” and “at least one.” As used in the specification and the claims, the singular form of “a,” “an,” and “the” include plural referents, such as unless the context clearly dictates otherwise. Additionally, Furthermore, as used herein, the terms “set” and “group” are intended to include one or more items (e.g., related items, unrelated items, a combination of related and unrelated items, etc.) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise. Further, the phrase “based on” is intended to mean “based at least partially on” unless explicitly stated otherwise. Further, the phrase “based on” may mean “in response to” and be indicative of a condition for automatically triggering a specified operation of an electronic device (e.g., a controller, a processor, a computing device, etc.) as appropriately referred to herein.

As used herein, the terms “communication” and “communicate” may refer to the reception, receipt, transmission, transfer, provision, and/or the like of information (e.g., data, signals, messages, instructions, commands, and/or the like). For one unit (e.g., a device, a system, a component of a device or system, combinations thereof, and/or the like) to be in communication with another unit means that the one unit is able to directly or indirectly receive information from and/or transmit information to the other unit. This may refer to a direct or indirect connection that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if at least one intermediary unit (e.g., a third unit located between the first unit and the second unit) processes information received from the first unit and communicates the processed information to the second unit. In some non-limiting embodiments, a message may refer to a network packet (e.g., a data packet and/or the like) that includes data. It will be appreciated that numerous other arrangements are possible.

It will be apparent that systems and/or methods, described herein, can be implemented in different forms of hardware, software, or a combination of hardware and software. The actual specialized control hardware or software code used to implement these systems and/or methods is not limiting of the implementations. Thus, the operation and behavior of the systems and/or methods are described herein without reference to specific software code, it being understood that software and hardware can be designed to implement the systems and/or methods based on the description herein.

Some non-limiting embodiments are described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, etc.

Provided are improved devices, systems, methods, and products for accurately identifying a characteristic of a medical device, such as a single-use medical device. Embodiments of the present disclosure may include a device that includes a medical device, a radio frequency identification (RFID) tear tag attached to the medical device, wherein the RFID tear tag comprises an RFID circuit and an electrical component electrically connected to the RFID circuit, wherein the electrical component comprises a first portion and a second portion, and wherein the first portion and the second portion are configured to be separable. In some non-limiting embodiments, when the electrical component is in a first state, the first portion and the second portion of the electrical component are electrically coupled, and the RFID circuit is configured to provide a first signal during interrogation by an RFID reader device, and when the electrical component is in a second state, the first portion and the second portion of the electrical component are not electrically coupled, and the RFID circuit is configured to provide a second signal during interrogation by an RFID reader device. In some non-limiting embodiments, the second signal is different from the first signal.

In some non-limiting embodiments, when the first portion and the second portion of the electrical component are electrically coupled, the first portion and the second portion of the electrical component are conductively coupled. In some non-limiting embodiments, the electrical component comprises an electrical conductor, which may include an electrically conductive trace. In some non-limiting embodiments, the device further comprises a base component, and the RFID circuit and the electrical component are positioned on the base component. In some non-limiting embodiments, the base component comprises a perforation, wherein the first portion and the second portion are configured to be separable based on the perforation of the base component. In some non-limiting embodiments, the first portion and the second portion of the electrical component are configured to be destructively disconnected when the electrical component is in the second state. In some non-limiting embodiments, the first portion and the second portion are configured to be separable independent of an additional mechanical component. In some non-limiting embodiments, the electrical component is a first electrical component, the device further comprises a second electrical component electrically connected to the RFID circuit, wherein the second electrical component comprises a first portion and a second portion, and wherein the first portion of the second electrical component and the second portion of the second electrical component are configured to be separable.

In this way, embodiments of the present disclosure provide an RFID tag that is able to provide accurate information with regard to the use of a medical device. For example, a user may be able to receive information from an RFID tag on the packaging of a medical device at the time that the packaging is removed from the medical device. In addition, a user may be able to read an RFID tag that is positioned on the medical device that would provide information regarding when the medical device was used. In this way, a reading from the RFID tag on the packaging may provide accurate information with regard to when the medical device is removed from the packaging and/or when the medical device is used.

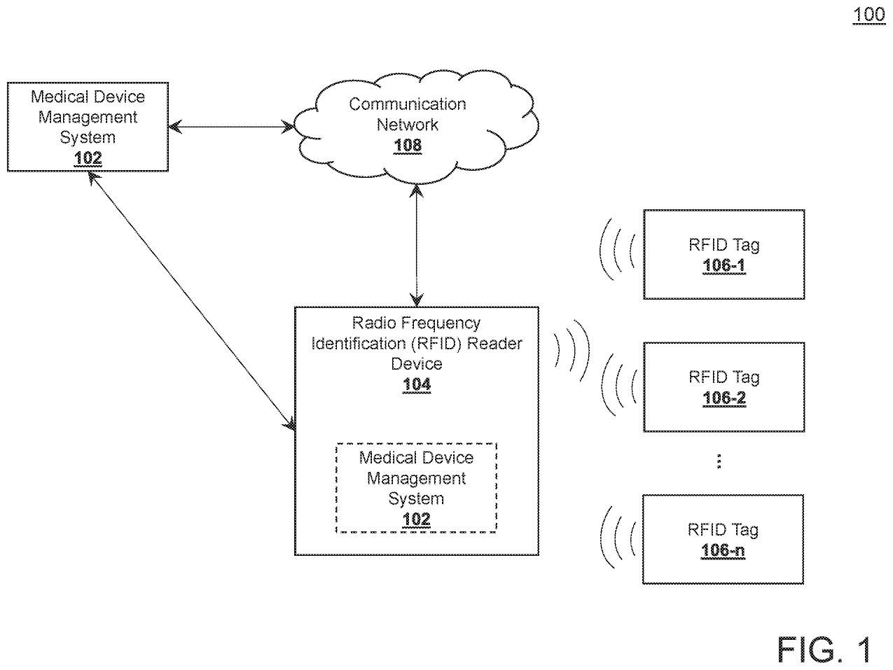

Referring now to FIG. 1, FIG. 1 is a diagram of an example environment 100 in which devices, systems, methods, and/or products described herein may be implemented. As shown in FIG. 1, environment 100 includes medical device management system 102, radio frequency identification (RFID) reader device 104, RFID tag 106-1 through RFID tag 106-n (which may be referred to collectively as RFID tags 106 and individually as RFID tag 106, where appropriate). Medical device management system 102 and RFID reader device 104 may interconnect (e.g., establish a connection to communicate, and/or the like) via wired connections, wireless connections, or a combination of wired and wireless connections.

Medical device management system 102 may include one or more devices configured to be in communication with RFID reader device 104 via communication network 108. For example, medical device management system 102 may include a server (e.g., a cloud server), a group of servers, a computing device, such as a mobile device (e.g., a smartphone) a tablet computer, a laptop computer, a desktop computer, and/or the like. In some non-limiting embodiments, medical device management system 102 may be configured to be in communication with RFID reader device 104 via a direct communication connection (e.g., a communication connection that is independent of a communication network, such as communication network 108), such as a short-range wireless communication connection (e.g., a near-field communication (NFC) communication connection, an RFID communication connection, a Bluetooth® communication connection, an infrared communication connection, etc.) or a wired communication connection (e.g., a connection that uses a cable and universal serial bus (USB) communication protocol). In some non-limiting embodiments, medical device management system 102 may be configured to determine a characteristic of a medical device based on data received from RFID reader device 104. In some non-limiting embodiments, medical device management system 102 may be a component of RFID reader device 104.

RFID reader device 104 may include one or more devices configured to be in communication with RFID tags 106. For example, RFID reader device 104 may include one or more RFID readers (e.g., a device that includes a radio frequency (RF) transmitter and an RF receiver that is capable of reading and/or writing information to an RFID tag). In some non-limiting embodiments, RFID reader device 104 may include one or more devices configured to be in communication with medical device management system 102 via communication network 108. For example, RFID reader device 104 may include a computing device, such as a mobile device (e.g., a smartphone, a wearable device, such as a wristlet, etc.), a tablet computer, a laptop computer, a desktop computer, and/or the like. In some non-limiting embodiments, RFID reader device 104 may be configured to be in communication with medical device management system 102 via a direct communication connection (e.g., a communication connection that is independent of a communication network, such as communication network 108), such as a short-range wireless communication connection (e.g., an NFC communication connection, an RFID communication connection, a Bluetooth® communication connection, an infrared communication connection, etc.) or a wired communication connection (e.g., a connection that uses a cable and universal serial bus (USB) communication protocol).

RFID tag 106 may include one or more RFID tags configured to provide a signal in response to an interrogation signal from an RFID reader, such as RFID reader device 104. In some non-limiting embodiments, RFID tag 106 may include a passive RFID tag or an active RFID tag. In some non-limiting embodiments, RFID tag 106 may include an RFID tear tag as described herein.

Communication network 108 may include one or more wired and/or wireless networks. For example, communication network 108 may include a cellular network (e.g., a long-term evolution (LTE) network, a third generation (3G) network, a fourth generation (4G) network, a fifth generation (5G), network a code division multiple access (CDMA) network, etc.), a public land mobile network (PLMN), a local area network (LAN), a wide area network (WAN), a metropolitan area network (MAN), a telephone network (e.g., the public switched telephone network (PSTN)), a private network, an ad hoc network, an intranet, the Internet, a fiber optic-based network, a cloud computing network, and/or the like, and/or a combination of some or all of these or other types of networks.

The number and arrangement of systems and/or devices shown in FIG. 1 are provided as an example. There may be additional systems and/or devices, fewer systems and/or devices, different systems and/or devices, or differently arranged systems and/or devices than those shown in FIG. 1. Furthermore, two or more systems and/or devices shown in FIG. 1 may be implemented within a single system or a single device, or a single system or a single device shown in FIG. 1 may be implemented as multiple, distributed systems or devices. Additionally or alternatively, a set of systems or a set of devices (e.g., one or more systems, one or more devices) of environment 100 may perform one or more functions described as being performed by another set of systems or another set of devices of environment 100.

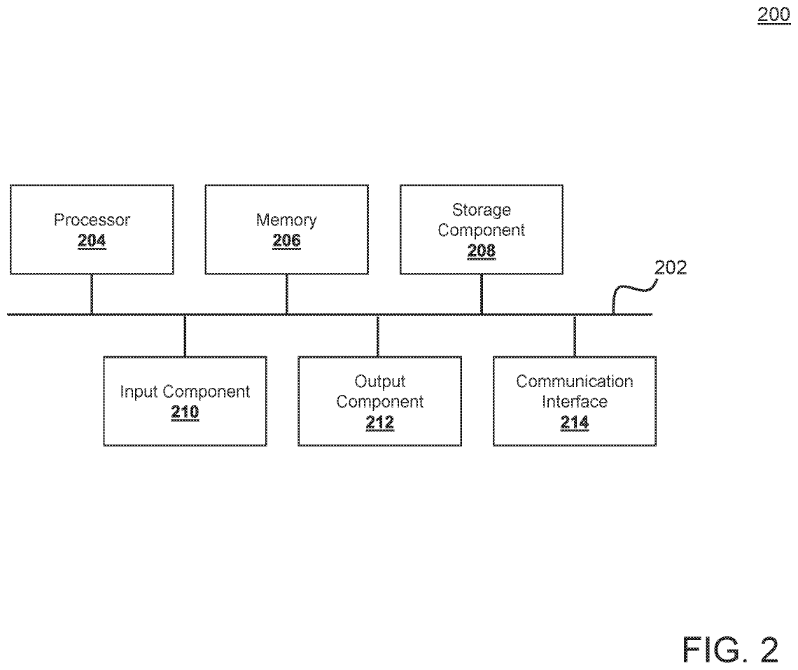

Referring now to FIG. 2, FIG. 2 is a diagram of example components of device 200. Device 200 may correspond to medical device management system 102 (one or more devices of medical device management system 102) and/or RFID reader device 104. In some non-limiting embodiments, medical device management system 102 and/or RFID reader device 104 may include at least one device 200 or at least one component of device 200. As shown in FIG. 2, device 200 may include bus 202, processor 204, memory 206, storage component 208, input component 210, output component 212, and communication interface 214.

Bus 202 may include a component that permits communication among the components of device 200. In some non-limiting embodiments, processor 204 may be implemented in hardware, software, or a combination of hardware and software. For example, processor 204 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function. Memory 206 may include random access memory (RAM), read-only memory (ROM), and/or another type of dynamic or static storage device (e.g., flash memory, magnetic memory, optical memory, etc.) that stores information and/or instructions for use by processor 204.

Storage component 208 may store information and/or software related to the operation and use of device 200. For example, storage component 208 may include a hard disk (e.g., a magnetic disk, an optical disk, a magneto-optic disk, a solid state disk, etc.), a compact disc (CD), a digital versatile disc (DVD), a floppy disk, a cartridge, a magnetic tape, and/or another type of computer-readable medium, along with a corresponding drive.

Input component 210 may include a component that permits device 200 to receive information, such as via user input (e.g., a touchscreen display, a keyboard, a keypad, a mouse, a button, a switch, a microphone, a camera, etc.). Additionally or alternatively, input component 210 may include a sensor for sensing information (e.g., a global positioning system (GPS) component, an accelerometer, a gyroscope, an actuator, etc.). Output component 212 may include a component that provides output information from device 200 (e.g., a display, a speaker, one or more light-emitting diodes (LEDs), etc.).

Communication interface 214 may include a transceiver-like component (e.g., a transceiver, a separate receiver and transmitter, etc.) that enables device 200 to communicate with other devices, such as via a wired connection, a wireless connection, or a combination of wired and wireless connections. Communication interface 214 may permit device 200 to receive information from another device and/or provide information to another device. For example, communication interface 214 may include an Ethernet interface, an optical interface, a coaxial interface, an infrared interface, a radio frequency (RF) interface, a universal serial bus (USB) interface, a Wi-Fi® interface, a cellular network interface, and/or the like.

Device 200 may perform one or more processes described herein. Device 200 may perform these processes based on processor 204 executing software instructions stored by a computer-readable medium, such as memory 206 and/or storage component 208. A computer-readable medium (e.g., a non-transitory computer-readable medium) is defined herein as a non-transitory memory device. A non-transitory memory device includes memory space located inside of a single physical storage device or memory space spread across multiple physical storage devices.

Software instructions may be read into memory 206 and/or storage component 208 from another computer-readable medium or from another device via communication interface 214. When executed, software instructions stored in memory 206 and/or storage component 208 may cause processor 204 to perform one or more processes described herein. Additionally or alternatively, hardwired circuitry may be used in place of or in combination with software instructions to perform one or more processes described herein. Thus, embodiments described herein are not limited to any specific combination of hardware circuitry and software.

Memory 206 and/or storage component 208 may include data storage or one or more data structures (e.g., a database, and/or the like). Device 200 may be capable of receiving information from, storing information in, communicating information to, or searching information stored in the data storage or one or more data structures in memory 206 and/or storage component 208. For example, the information may include data associated with a real-time mobile device application profile, data associated with a historical mobile device application profile, input data, output data, transaction data, account data, or any combination thereof.

The number and arrangement of components shown in FIG. 2 are provided as an example. In some non-limiting embodiments, device 200 may include additional components, fewer components, different components, or differently arranged components than those shown in FIG. 2. Additionally or alternatively, a set of components (e.g., one or more components) of device 200 may perform one or more functions described as being performed by another set of components of device 200.

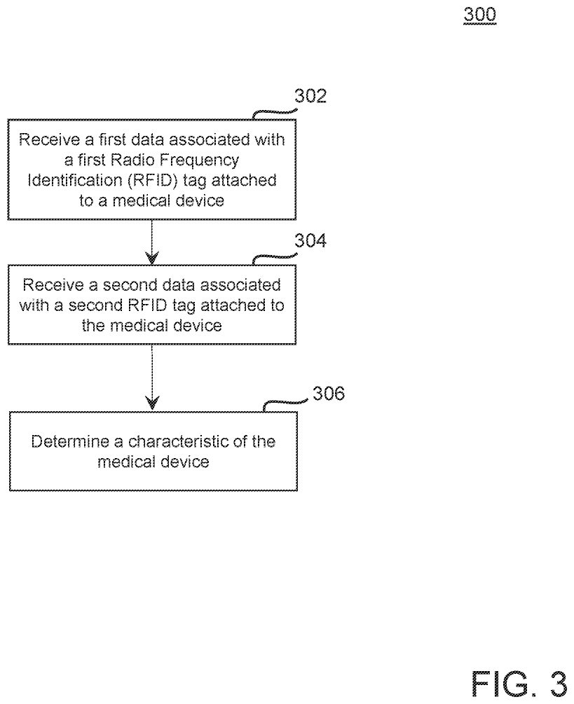

Referring now to FIG. 3, FIG. 3 is a flowchart of a non-limiting embodiment of a process 300 for determining a characteristic of a medical device. In some non-limiting embodiments, one or more of the functions described with respect to process 300 may be performed (e.g., completely, partially, etc.) by medical device management system 102. In some non-limiting embodiments, one or more of the steps of process 300 may be performed (e.g., completely, partially, and/or the like) by another device or a group of devices separate from medical device management system 102, such as RFID reader device 104.

As shown in FIG. 3, at step 302, process 300 may include receiving first data associated with a first radio frequency identification (RFID) tag attached to a medical device. For example, medical device management system 102 may receive (e.g., receive in real-time) the first data associated with RFID tag 106-1 attached to (e.g., applied to, adhered to, stuck on, etc.) a surface of the medical device (e.g., a container, a syringe, a catheter, etc.).

In some non-limiting embodiments, data associated with RFID tag 106 (e.g., first data associated with RFID tag 106-1, second data associated with RFID tag 106-2, additional data associated with RFID tag 106-n, etc.) may include data associated with an identification of an individual on which the medical device is to be used, data associated with a substance (e.g., a medication, a medicine, a drug, etc.), such as a type of substance, with which the medical device is filled, data associated with an amount of substance with which the medical device is filled, data associated with a time at which the medical device was filled with a substance, data associated with a time at which the medical device was placed (e.g., placed initially, replaced, etc.) in packaging (e.g., a sterile packaging), data associated with a time at which the medical device was removed from packaging, data associated with a time at which the medical device was placed in an active state (e.g., a ready to use state, an open state, a state at which a substance in the medical device is to be administered, etc.), data associated with a time at which the medical device was placed in an inactive state (e.g., a not ready to use state, a closed state, a state at which a substance in the medical device is not to be administered, etc.), data associated with a time at which the medical device began to be used in an active state (e.g., a state at which a substance in the medical device began to be administered), data associated with a time at which the medical device finished being used in an active state (e.g., a state at which a substance in the medical device is finished being administered), data associated with a time at which one or more components (e.g., one or more safety components) of the medical device were placed in a specific position (e.g., adjacent to each other, in a coupled connection, in a fixed connection, etc.) and/or the like.

In some non-limiting embodiments, RFID tag 106 may provide (e.g., transmit) the data associated with RFID tag 106 (e.g., a signal including the data) based on receiving an interrogation signal from RFID reader device 104. In some non-limiting embodiments, RFID tag 106 may provide (e.g., automatically provide) the data associated with RFID tag 106 continuously, periodically, or at a predetermined time interval.

In some non-limiting embodiments, RFID tag 106 may include an RFID circuit and an electrical component electrically connected to the RFID circuit. In some non-limiting embodiments, when the electrical component is in a first state, the RFID circuit is configured to provide data associated with RFID tag 106. In some non-limiting embodiments, when the electrical component is in a second state, the RFID circuit is configured to provide additional data associated with RFID tag 106. In some non-limiting embodiments, when the electrical component is in the first state, the RFID circuit is configured to provide data associated with RFID tag 106 that is different from the data associated with RFID tag 106 that the RFID circuit is configured to provide when the electrical component is in the second state.

In some non-limiting embodiments, medical device management system 102 may receive the first data associated with RFID tag 106-1 from RFID reader device 104. For example, RFID reader device 104 may read RFID tag 106-1 and obtain the first data associated with RFID tag 106-1 based on reading RFID tag 106-1. RFID reader device 104 may transmit the first data associated with RFID tag 106-1 to medical device management system 102, and medical device management system 102 may receive the first data associated with RFID tag 106-1 from RFID reader device 104 based on RFID reader device 104 reading RFID tag 106-1.

In some non-limiting embodiments, a medical device may include a single-use medical device. A single-use medical device may include a medical device that designed and configured to be used once and then disposed of (e.g., discarded). Additionally or alternatively, a single-use medical device may include a medical device that designed and configured to be used once on a single patient. In some non-limiting embodiments, a medical device, such as a single-use medical device, may include an indication (e.g., an indicia, such as a sign, a sticker, a marking, etc.) that the single-use medical device is to be used once and then disposed of. In some non-limiting embodiments, a medical device may include a container, such as a single-use container.

As shown in FIG. 3, at step 304, process 300 may include receiving second data associated with a second RFID tag attached to the medical device. For example, medical device management system 102 may receive the second data associated with RFID tag 106-2 attached to the medical device. In some non-limiting embodiments, the first data associated with RFID tag 106-1 may be different from the data associated with RFID tag 106-2.

In some non-limiting embodiments, medical device management system 102 may receive the second data associated with RFID tag 106-2 from RFID reader device 104. For example, RFID reader device 104 may read RFID tag 106-2 and obtain the second data associated with RFID tag 106-2 based on reading RFID tag 106-2. RFID reader device 104 may transmit the second data associated with RFID tag 106-2 to medical device management system 102, and medical device management system 102 may receive the second data associated with RFID tag 106-2 from RFID reader device 104 based on RFID reader device 104 reading RFID tag 106-2.

As shown in FIG. 3, at step 306, process 300 may include determining a characteristic of the medical device. For example, medical device management system 102 may determine a characteristic of a medical device based on the first data associated with RFID tag 106-1 attached to the medical device and/or the second data associated with RFID tag 106-2 attached to the medical device. In some non-limiting embodiments, medical device management system 102 may determine the characteristic of the medical device by comparing the first data to the second data. In some non-limiting embodiments, medical device management system 102 may determine the characteristic of the medical device by comparing data associated with RFID tag 106 provided by an RFID circuit of RFID tag 106 when an electrical component of RFID tag 106 is in a first state to data associated with RFID tag 106 provided by an RFID circuit of RFID tag 106 when an electrical component of RFID tag 106 is in a second state.

In some non-limiting embodiments, medical device management system 102 may determine an identification of an individual on which the medical device is to be used, a time at which the medical device was filled with a substance, a time at which the medical device was placed in packaging, a time at which the medical device was removed from packaging, a time at which the medical device was placed in an active state, a time at which the medical device was placed in an inactive state, a time at which the medical device began to be used in an active state, a time at which the medical device finished being used in an active state, a time at which one or more components of the medical device were placed in a specific position, and/or the like.

Additionally or alternatively, medical device management system 102 may determine a type of a substance administered using the medical device, an amount of a substance administered using the medical device, a time of administration of a substance administered using the medical device, a number of times the medical device has been used (e.g., reused), a number of times the medical device has been placed in an active state, a number of times the medical device has been placed in an inactive state, a number of times that the medical device has been successfully used with (e.g., successfully applied to, successfully inserted into a body of, successfully delivered to, etc.) an individual (e.g., a patient), a number of times that the medical device has not been successfully used with an individual, a number of times that the medical device was attempted to be used with individual, a number of times that the medical device was attempted to be used with individual but was not ultimately used, and/or the like.

In some non-limiting embodiments, medical device management system 102 may assign a medical device to an individual (e.g., a patient) based on data associated with RFID tag 106 attached to the medical device. For example, medical device management system 102 may assign the medical device to the individual based on data associated with an identification of the individual. In such an example, medical device management system 102 may determine whether the medical device was used (e.g., appropriately used) on the individual based on additional data associated with RFID tag 106 attached to the medical device. In some non-limiting embodiments, medical device management system 102 may receive the additional data from RFID reader device 104 after the medical device was assigned to the individual, and medical device management system 102 may determine whether the medical device was used on the individual.

In some non-limiting embodiments, medical device management system 102 may perform an action based on data associated with RFID tag 106 attached to the medical device. For example, medical device management system 102 may transmit the data to a database. In some non-limiting embodiments, medical device management system 102 may transmit the data to an electronic medical record (EMR) system for automatic logging and/or safety confirmation that medication to be administered matches a medication order for an individual.

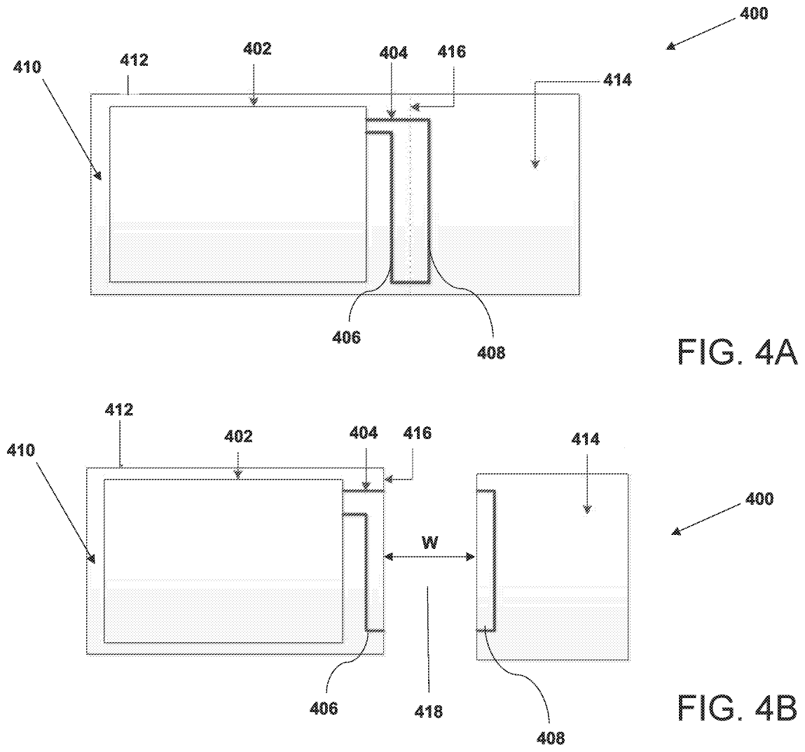

Referring to FIGS. 4A and 4B, FIGS. 4A and 4B are diagrams of RFID tag 400 and components thereof. In some non-limiting embodiments, RFID tag 400 may be the same as or similar to RFID tag 106. In some non-limiting embodiments, RFID tag 400 may be attached to a medical device. For example, RFID tag 400 may be attached to a syringe, an IV assembly, a catheter, a container, medication packaging and/or the like.

In some non-limiting embodiments, RFID tag 400 may be attached to a syringe (e.g., an unfilled syringe, prefilled syringe, needleless syringe, and/or the like). For example, RFID tag 400 may be attached to a syringe cap of the syringe. In such an example, RFID tag 400 may bridge a gap between the syringe cap and the syringe body of the syringe. As another example, RFID tag 400 may be attached to a safety device of the syringe. As another example, RFID tag 400 may be attached to a barrel and to a plunger of the syringe. In such an example, RFID tag 400 may bridge a gap between a barrel and a plunger of the syringe. As another example, RFID tag 400 may be attached to a collar (e.g., a collapsible collar or retractable collar) and/or a connector (e.g., luer connector and/or the like) of a needleless syringe. In such an example, RFID tag 400 may be attached to a collar and/or connector of the needless syringe at one end and to a mating device (e.g., needle, tubing, luer connector and/or the like) at the opposite end.

In some non-limiting embodiments, RFID tag 400 may be attached to an IV assembly. For example, RFID tag 400 may be positioned between two components of an IV assembly. For example, RFID tag 400 may be positioned between a syringe (e.g., a needleless syringe) and a connector (e.g., an IV connector). In some non-limiting embodiments, RFID tag 400 may be attached to two components of an IV assembly. For example, RFID tag 400 may be attached to a syringe (e.g., a needleless syringe) and/or a connector (e.g., an IV connector).

In some non-limiting embodiments, RFID tag 400 may be attached to a catheter. For example, RFID tag 400 may be attached to a safety device of a catheter. As another example, RFID tag 400 may be attached to a cap (e.g., disinfecting cap) of the catheter. As another example, RFID tag 400 may be attached to a connector (e.g., luer connector and/or the like) of the catheter. In such an example, RFID tag 400 may be attached to a connector of the catheter at one end and to a mating device (e.g., needleless connector, manifold, catheter line, syringe, and/or the like) at the opposite end.

In some non-limiting embodiments, RFID tag 400 may be attached to a container. For example, RFID tag 400 may be attached to a medication container (e.g., medication bottles, medication boxes, such as pill boxes, IV bags, and/or the like). In such an example, RFID tag 400 may be attached to a cap of a container (e.g., a medication container). For example, RFID tag 400 may bridge a gap between the cap of the container and the body of the container. In some non-limiting embodiments, RFID tag 400 may be attached to a food and/or beverage container. In such an example, RFID tag 400 may be attached to a cap of a food and/or beverage container.

In some non-limiting embodiments, RFID tag 400 may be attached to a packaging for a medical device. In such an example, RFID tag 400 may be attached to a lid and/or a cap of a packaging for a medical device. For example, RFID tag 400 may bridge a gap between the lid and/or cap of a packaging for a medical device and a body of a packaging of a medical device. In some non-limiting embodiments, RFID tag 400 may be attached to a medication packaging (e.g., a medication blister pack). In such an example, RFID tag 400 may be provided on the label of the medication packaging.

In some non-limiting embodiments, RFID tag 400 may have a size that allows RFID tag 400 to be attached to a particular medical device. In such an example, the size of RFID tag 400 may be proportional to the size of the medical device or component of the medical device that RFID tag 400 is attached to. As another example, RFID tag 400 may have a size that is sufficient to include each of the components of RFID tag 400 that are to be positioned on RFID tag 400.

In some non-limiting embodiments, RFID tag 400 may have a shape that allows RFID tag 400 to be attached to a particular medical device. In such an example, RFID tag 400 may have a shape that corresponds to an area of a medical device that RFID tag 400 is attached to. As another example, RFID tag 400 may have a shape that is sufficient to include each of the components of RFID tag 400 that are to be positioned on RFID tag 400.

In some non-limiting embodiments, RFID tag 400 may transmit a signal to an RFID reader device (e.g., RFID reader device 104). In some non-limiting embodiments, RFID tag 400 may be a passive RFID tag. In such an example, RFID tag 400 (e.g., RFID circuit 402 of RFID tag 400) may be powered by electromagnetic energy from an electromagnetic wave transmitted by an RFID reader device to interrogate an RFID tag 400. In some non-limiting embodiments, RFID tag 400 may be an active RFID tag. In such an example, RFID tag 400 may be powered by a power source included in RFID tag 400 and, thus, can be read at a greater range by an RFID reader device compared to a passive RFID tag. In some non-limiting embodiments, RFID tag 400 may be read by an RFID reader device at a distance of at least 100 meters. In some non-limiting embodiments, RFID tag 400 may include an NFC tag. In some non-limiting embodiments, RFID tag 400 may be an RFID tear tag 400. RFID tear tag 400, as used herein, refers to an RFID tag that includes an electrical component, where the output of RFID tag 400 depends on if the electrical component is in a closed state or an open state.

In some non-limiting embodiments, as shown in FIGS. 4A and 4B, RFID tag 400 may include RFID circuit 402, electrical component 404, and base component 410. In some non-limiting embodiments, RFID circuit 402 may be positioned on base component 410. In such an example, RFID circuit 402 may be attached to base component 410 (e.g., via an adhesive, welding, soldering, and/or the like). In some non-limiting embodiments, electrical component 404 may be positioned on base component 410. For example, electrical component 404 may be attached to base component 410 (e.g., via an adhesive, welding, soldering, and/or the like). As another example, electrical component 404 may be embedded into base component 410. In some non-limiting embodiments, electrical component 404 may be coupled to RFID circuit 402. For example, electrical component 404 may be electrically connected to RFID circuit 402.

In some non-limiting embodiments, base component 410 may include attachment point 416 that is configured to allow for base component 410 to be separated into a first section and a second section.

In some non-limiting embodiments, RFID tag 400 may include RFID circuit 402. In some non-limiting embodiments, RFID circuit 402 may have a size that is appropriate based on an application for which RFID tag 400 is designed. For example, RFID circuit 402 may have a size such that RFID circuit 402 may be positioned on base component 410. In some non-limiting embodiments, RFID circuit 402 may include various components typically provided in RFID circuits. In some non-limiting embodiments, RFID circuit 402 may include a radio transponder, a radio receiver, a radio transmitter and/or the like. In some non-limiting embodiments, RFID circuit 402 may include a power source (e.g., a battery and/or the like). In some non-limiting embodiments, RFID circuit 402 may be powered by electromagnetic energy. As used herein, electromagnetic energy refers to energy from electromagnetic waves characterized by the electromagnetic spectrum. In such an example, RFID circuit 402 may be powered by electromagnetic energy from an RFID reader device.

In some non-limiting embodiments, RFID circuit 402 may provide a signal to an RFID reader device. In such an example, RFID circuit 402 may provide a signal during interrogation by an RFID reader device. In some non-limiting embodiments, RFID circuit 402 may provide more than one signal.

In some non-limiting embodiments, RFID circuit 402 may provide a signal based on a state of one or more components of RFID tag 400. For example, RFID circuit 402 may provide a first signal when one or more components of RFID tag 400 are in a first state, and provide a second signal when one or more components of RFID tag 400 are in a second state.

In some non-limiting embodiments, electrical component 404 may include a conductive element, such as a wire, an electrical trace (e.g., an electrically conductive trace), an electrical conductor, a metallic strip, a conductive ink, a conductive coating, conductive tabs, and/or the like. In some non-limiting embodiments, electrical component 404 may have a size that is appropriate based on an application for which RFID tag 400 is designed. For example, electrical component 404 may have a size such that electrical component 404 may be positioned on base component 410. In some non-limiting embodiments, electrical component 404 may include a conductive material.

In some non-limiting embodiments, electrical component 404 may include a first portion 406 and a second portion 408. In some non-limiting embodiments, first portion 406 and second portion 408 of electrical component 404 may be positioned on base component 410. For example, first portion 406 and second portion 408 of electrical component 404 may be attached to base component 410 (e.g., via an adhesive, welding, soldering, and/or the like). As another example, first portion 406 and second portion 408 of electrical component 404 may be embedded into base component 410.

In some non-limiting embodiments, first portion 406 and second portion 408 of electrical component 404 are separable. For example, first portion 406 and second portion 408 may be destructively separable. In such an example, destructively separable means that once first portion 406 and second portion 408 of electrical component 404 are decoupled, first portion 406 and second portion 408 cannot be recoupled. In another example, first portion 406 and second portion 408 of electrical component 404 may be non-destructively separable. In such an example, non-destructively separable means that once first portion 406 and second portion 408 of electrical component 404 are decoupled, first portion 406 and second portion 408 of electrical component 404 may be recoupled. In some non-limiting embodiments, first portion 406 and second portion 408 of electrical component 404 may be configured to be separable independent of an additional mechanical component.

In some non-limiting embodiments, first portion 406 and second portion 408 of electrical component 404 may form continuous, unitary electrical component 404. In some non-limiting embodiments, first portion 406 and second portion 408 of electrical component 404 may be coupled. For example, first portion 406 and second portion 408 of electrical component 404 may be electrically coupled (e.g., conductively coupled). In some non-limiting embodiments, first portion 406 of electrical component 404 may be connected (e.g., electrically connected) to RFID circuit 402.

In some non-limiting embodiments, base component 410 may be constructed of a material that is appropriate based on an application for which RFID tag 400 is designed. In some non-limiting embodiments, base component 410 may have a shape that is appropriate based on an application for which RFID tag 400 is designed. In such an example, a shape of base component 410 is not particularly limited, so long as the shape of base component 410 may fit the required components positioned thereon. Non-limiting examples of the shape of base component 410 include, but are not limited to, a rectangle, a circle, an oval, a square, a parallelogram, a trapezoid, triangle, and/or the like. In some non-limiting embodiments, base component 410 may include a material that is appropriate based on an application for which RFID tag 400 is designed. In some non-limiting embodiments, base component 410 may include paper, plastic, and/or the like.

In some non-limiting embodiments, base component 410 may include first portion 412 and second portion 414. In some non-limiting embodiments, RFID circuit 402 may be positioned on first portion 412 of base component 410. For example, RFID circuit 402 may be attached to first portion 412 of base component 410 (e.g., via an adhesive, welding, soldering, and/or the like). In some non-limiting embodiments, first portion 406 of electrical component 404 may be positioned on first portion 412 of base component 410. For example, first portion 406 of electrical component 404 may be attached to first portion 412 of base component 410 (e.g., via an adhesive, welding, soldering, and/or the like). As another example, first portion 406 of electrical component 404 may be embedded into first portion 412 of base component 410. In some non-limiting embodiments, second portion 408 of electrical component 404 may be positioned on second portion 414 of base component 410. For example, second portion 408 of electrical component 404 may be attached to second portion 414 of base component 410 (e.g., via an adhesive, welding, soldering, and/or the like). In another example, second portion 408 of electrical component 404 may be embedded into second portion 414 of base component 410.

In some non-limiting embodiments, base component 410 may include attachment point 416 that separates first portion 412 of base component 410 from second portion 414 of base component 410. In some non-limiting embodiments, attachment point 416 may include a perforation. In such an example, a perforation may include a plurality of holes that span the length of attachment point 416. In some non-limiting embodiments, attachment point 416 includes a perforation provided on one side of base component 410. In some non-limiting embodiments, attachment point 416 may include a perforation provided on only one side of base component 410. For example, the perforation of attachment point 416 may be provided on the side of base component 410 in which RFID circuit 402 and electrical component 404 are positioned on. In another example, the perforation of attachment point 416 may be provided on the opposite side of base component 410 in which RFID circuit 402 and electrical component 404 are positioned on. In another non-limiting embodiment, the perforation of attachment point 416 may be provided on both sides of base component 410.

In some non-limiting embodiments, attachment point 416 may include a creased area. In such an example, the creased area of attachment point 416 may include a thinner area, or otherwise mechanically inferior area compared to the rest of base component 410. In some non-limiting embodiments, attachment point 416 may include an adhesive. In such an example, the adhesive of attachment point 416 may be configured to couple first portion 412 of base component 410 to second portion 414 of base component 410. In some non-limiting embodiments, attachment point 416 may include a plurality of magnets. In such an example, first portion 412 of base component 410 may include at least one magnet at attachment point 416, and second portion 414 of base component 410 may include at least one magnet at attachment point 416, such that the magnets may couple first portion 412 of base component 410 to second portion 414 of base component 410.

In some non-limiting embodiments, first portion 412 and second portion 414 of base component 410 may form continuous, unitary base component 410 at attachment point 416. In such an example, first portion 412 of base component 410 is materially connected to second portion 414 of base component 410 at attachment point 416. In some non-limiting embodiments, first portion 412 of base component 410 may be separate from second portion 414 of base component 410 at attachment point 416, such that first portion 412 and second portion 414 of base component 410 are not materially connected, but are coupled at attachment point 416.

In some non-limiting embodiments, first portion 406 and second portion 408 of electrical component 404 may be separable at attachment point 416 of base component 410. In such an example, first portion 406 of electrical component 404 may be coupled to second portion 408 of electrical component 404 at attachment point 416 of base component 410.

Referring to FIG. 4A, in some non-limiting embodiments, first portion 406 and second portion 408 of electrical component 404 may be coupled together when electrical component 404 is in a first state. For example, first portion 406 and second portion 408 of electrical component 404 may be electrically coupled (e.g., conductively coupled) together when electrical component 404 is in a first state. In some non-limiting embodiments, first portion 412 and second portion 414 of base component 410 may be coupled together, such that first portion 406 and second portion 408 of electrical component 404 are coupled together, when electrical component 404 is in a first state.

In some non-limiting embodiments, first portion 406 and second portion 408 of electrical component 404 may be recoupled after previously being decoupled when electrical component 404 is in the first state. For example, first portion 406 and second portion 408 of electrical component 404 may be electrically recoupled (e.g., conductively recoupled) after previously being electrically decoupled (e.g., conductively decoupled) when electrical component 404 is in a first state. In some non-limiting embodiments, first portion 412 and second portion 414 of base component 410 may be recoupled after previously being decoupled, such that first portion 406 and second portion 408 of electrical component 404 are recoupled after previously being decoupled, when electrical component 404 is in a first state. In some non-limiting embodiments, first portion 406 and second portion 408 of electrical component 404 may be decoupled and then recoupled to change the state of electrical component 404 from the first state, to the second state, and back to the first state and to change the signal provided by RFID circuit 402 from the first signal, to the second signal, and back to the first signal any number of times.

In some non-limiting embodiments, RFID circuit 402 may be configured to provide a first signal, such as during interrogation by an RFID reader device, when electrical component 404 is in a first state. In some non-limiting embodiments, a first signal provided by RFID circuit 402 may include data associated with an indication of a first state of a medical device. In some non-limiting embodiments, data associated with a first state of a medical device may indicate a closed configuration. For example, data associated with a first state of a medical device may indicate a closed configuration for the medical device. In such an example, data associated with a first state of a medical device may indicate that a cap and/or lid of a medical device (e.g. container and/or the like) is secured to a body of the medical device to seal the contents (e.g., medication) within the medical device.

In some non-limiting embodiments, data associated with a first state of a medical device may indicate an unused configuration. For example, data associated with a first state of a medical device may indicate an unused configuration for the medical device. For example, data associated with a first state of a medical device may indicate that a medication has not been delivered by the medical device (e.g., syringe and/or the like). In another example, data associated with a first state of a medical device may indicate that a safety device of the medical device (e.g., syringe, catheter, and/or the like) has not been activated. In another example, data associated with a first state of a medical device may indicate that a collar (e.g., collapsible collar, retractable collar, and/or the like) of the medical device has not been displaced in order to connect the medical device to a mating device.

In some non-limiting embodiments, data associated with a first state of a medical device may indicate a coupled configuration. For example, data associated with a first state of a medical device may indicate a coupled configuration for a medical device. For example, data associated with a first state of a medical device may indicate that a first component of a medical device is coupled to a second component of a medical device. In such an example, data associated with a first state of a medical device may indicate that a first connector (e.g., a luer connector and/or the like) is connected to a second connector (e.g., a luer connector and/or the like), such as, for example, the connection between a needleless syringe and an IV connector.

In some non-limiting embodiments, data associated with a first state of a medical device may indicate an uncontaminated configuration. For example, data associated with a first state of a medical device may indicate an uncontaminated configuration of a medical device. For example, data associated with a first state of a medical device may indicate that a floating collar of the medical device has not been bumped or touched prior to connection (e.g., luer attachment and/or the like). In another example, data associated with a first state of a medical device may indicate that a deformable element in a collar of a medical device (e.g., a syringe and/or the like) has not been deformed.

In some non-limiting embodiments, a first signal provided by RFID circuit 402 may include data associated with an indication of a first state of a packaging of a medical device. In some non-limiting embodiments, data associated with a first state of a packaging of a medical device may indicate a sealed configuration. For example, data associated with a first state of a packaging of a medical device may indicate a sealed configuration of a packaging of a medical device. In such an example, data associated with a first state of a packaging of a medical device may indicate that the packaging has yet to be open to remove the medical device from the packaging. In another example, data associated with a first state of a packaging of a medical device may indicate that a seal holding a medication in the packaging has not been broken (e.g., a medication blister pack and/or the like).

Referring to FIG. 4B, in some non-limiting embodiments, first portion 406 and second portion 408 of electrical component 404 may be decoupled when electrical component 404 is in a second state. For example, first portion 406 and second portion 408 of electrical component 404 may not be electrically coupled (e.g., not conductively coupled) when electrical component 404 is in a second state. In some non-limiting embodiments, first portion 412 of base component 410 may be decoupled from second portion 414 of base component 410 at attachment point 416, such that first portion 406 and second portion 408 of electrical component 404 are decoupled, when electrical component 404 is in a second state. For example, first portion 412 of base component 410 may be decoupled from second portion 414 of base component 410 at attachment point 416, such that there is gap 418 between first portion 412 and second portion 414 of base component 410. Width W of gap 418 is not particularly limited, so long as width W is sufficiently wide, such that first portion 406 and second portion 408 of electrical component 404 are not electrically coupled. In some non-limiting embodiments, first portion 412 of base component 410 may be decoupled from second portion 414 of base component 410 by pulling and/or rotating first portion 412 of base component 410 away from second portion 414 of base component 410 at attachment point 416, such as to form gap 418 with width W between first portion 412 and second portion 414 of base component 410. For example, base component 410 may be broke at attachment point 416 and then first portion 412 of base component 410 may be pulled and/or rotated away from second portion 414 of base component 410. In some non-limiting embodiments, RFID circuit 402 may be configured to provide a second signal, such as during interrogation by an RFID reader device, when electrical component 404 is in a second state. In some non-limiting embodiments, the second signal provided by RFID circuit 402 may be different from the first signal provided by RFID circuit 402.

In some non-limiting embodiments, a second signal provided by RFID circuit 402 may include data associated with an indication of a second state of a medical device. In some non-limiting embodiments, data associated with a second state of a medical device may indicate an open configuration. For example, data associated with a second state of a medical device may indicate an open configuration for the medical device. In such an example, data associated with a second state of a medical device may indicate that a cap and/or lid of a medical device (e.g. container and/or the like) has been removed from a body of the medical device, such that contents (e.g., medication) within the medical device are no longer sealed within the medical device.

In some non-limiting embodiments, data associated with a second state of a medical device may indicate an unfilled configuration. For example, data associated with a second state of a medical device may indicate an unfilled configuration for the medical device. For example, data associated with a second state of a medical device may indicate that a medical device (e.g., syringe and/or the like) has yet to receive a medication.

In some non-limiting embodiments, data associated with a second state of a medical device may indicate a used configuration. For example, data associated with a second state of a medical device may indicate a used configuration for the medical device. For example, data associated with a second state of a medical device may indicate that a medication has been delivered by the medical device (e.g., syringe and/or the like). In another example, data associated with a second state of a medical device may indicate that a safety device of the medical device (e.g., syringe, catheter, and/or the like) has been activated. In another example, data associated with a second state of a medical device may indicate that a collar (e.g., collapsible collar, retractable collar, and/or the like) of the medical device has been displaced in order to connect the medical device to a mating device.

In some non-limiting embodiments, data associated with a second state of a medical device may indicate a decoupled configuration. For example, data associated with a second state of a medical device may indicate a decoupled configuration for a medical device. For example, data associated with a second state of a medical device may indicate that a first component of a medical device is not coupled to a second component of a medical device. In such an example, data associated with a second state of a medical device may indicate that a first connector (e.g., a luer connector and/or the like) is not connected to a second connector (e.g., a luer connector and/or the like), such as, for example, the connection between a needleless syringe and an IV connector.

In some non-limiting embodiments, data associated with a second state of a medical device may indicate a contaminated configuration. For example, data associated with a second state of a medical device may indicate a contaminated configuration of a medical device. For example, data associated with a second state of a medical device may indicate that a floating collar of the medical device has been bumped or touched prior to connection (e.g., luer attachment and/or the like). In another example, data associated with a second state of a medical device may indicate that a deformable element in a collar of a medical device (e.g., a syringe and/or the like) has been deformed.

In some non-limiting embodiments, a second signal provided by RFID circuit 402 may include data associated with an indication of a second state of a packaging of a medical device. In some non-limiting embodiments, data associated with a second state of a packaging of a medical device may indicate an unsealed configuration. For example, data associated with a second state of a packaging of a medical device may indicate an unsealed configuration of a packaging of a medical device. In such an example, data associated with a second state of a packaging of a medical device may indicate that the packaging has been opened to remove the medical device from the packaging. In another example, data associated with a second state of a packaging of a medical device may indicate that a seal holding a medication in the packaging has been broken (e.g., a medication blister pack and/or the like).

In some non-limiting embodiments, first portion 406 and second portion 408 of electrical component 404 may be recoupled after previously being decoupled when electrical component 404 is in the third state. For example, first portion 406 and second portion 408 of electrical component 404 may be electrically recoupled (e.g., conductively recoupled) after previously being electrically decoupled (e.g., conductively decoupled) when electrical component 404 is in a third state. In some non-limiting embodiments, first portion 412 and second portion 414 of base component 410 may be recoupled after previously being decoupled, such that first portion 406 and second portion 408 of electrical component 404 are recoupled after previously being decoupled, when electrical component 404 is in a third state. In some non-limiting embodiments, first portion 406 and second portion 408 of electrical component 404 may be decoupled and then recoupled to change the state of electrical component 404 from the first state, to the second state, to the third state and to change the signal provided by RFID circuit 402 from the first signal, to the second signal, to the third signal any number of times to provide any number of signals.

In some non-limiting embodiments, RFID circuit 402 may be configured to provide a third signal, such as during interrogation by an RFID reader device, when electrical component 404 is in a third state. In some non-limiting embodiments, the third signal provided by RFID circuit 402 may be different from both the first signal provided by RFID circuit 402 and the second signal provided by RFID circuit 402. In some non-limiting embodiments, a third signal provided by RFID circuit 402 may include data associated with an indication of a third state of a medical device. In some non-limiting embodiments, data associated with a third state of a medical device may indicate a reclosed configuration. For example, data associated with a third state of a medical device may indicate a reclosed configuration for the medical device. In such an example, data associated with a third state of a medical device may indicate that a cap and/or lid of a medical device (e.g. container and/or the like) has been reattached to a body of the medical device after the cap and/or lid had been previously removed to unseal the contents (e.g., medication) within the medical device.

In some non-limiting embodiments, data associated with a third state of a medical device may indicate a reused configuration. For example, data associated with a third state of a medical device may indicate a reused configuration for the medical device. For example, data associated with a third state of a medical device may indicate that a medical device (e.g., syringe and/or the like) has been refilled with medication after previously delivering a medication. In another example, data associated with a third state of a medical device may indicate that a safety device of the medical device (e.g., syringe, catheter, and/or the like) has been disabled after previously being activated. In another example, data associated with a third state of a medical device may indicate that a collar (e.g., collapsible collar, retractable collar, and/or the like) of the medical device has returned to the original position of the collar after previously being displaced in order to connect the medical device to a mating device.

In some non-limiting embodiments, data associated with a third state of a medical device may indicate a recoupled configuration. For example, data associated with a third state of a medical device may indicate a recoupled configuration for a medical device. For example, data associated with a third state of a medical device may indicate that a first component of a medical device is recoupled to a second component of a medical device after the first component had previously being decoupled from the second component. In such an example, data associated with a third state of a medical device may indicate that a first connector (e.g., a luer connector and/or the like) is reconnected and/or re-secured to a second connector (e.g., a luer connector and/or the like) after the first connector had previously been disconnected from the second connector, such as, for example, the connection between a needleless syringe and an IV connector.

In some non-limiting embodiments, a third signal provided by RFID circuit 402 may include data associated with an indication of a third state of a packaging of a medical device. In some non-limiting embodiments, data associated with a third state of a packaging of a medical device may indicate a resealed configuration. For example, data associated with a third state of a packaging of a medical device may indicate a resealed configuration of a packaging of a medical device. In such an example, data associated with a third state of a packaging of a medical device may indicate that the packaging has been resealed after previously being opened to remove the medical device from the packaging. In another example, data associated with a third state of a packaging of a medical device may indicate that a seal holding a medication in the packaging has been resealed after previously being broken (e.g., a medication blister pack and/or the like).

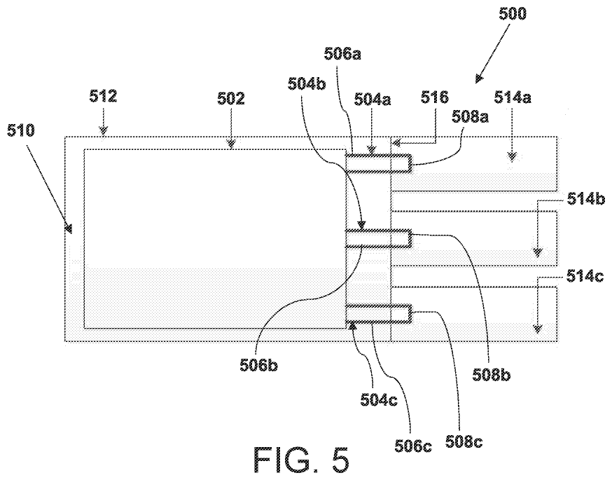

Referring to FIG. 5, FIG. 5 is diagram of RFID tag 500 and components thereof. In some non-limiting embodiments, RFID tag 500 may be the same as or similar to RFID tag 400 and/or RFID tag 106. In some non-limiting embodiments, RFID tag 500 may include a plurality of electrical components 504a, 504b, 504c, and base component 510 may include a plurality of separable portions 514a, 514b, 514c to correspond to each of the plurality of electrical components 504a, 504b, 504c.

In some non-limiting embodiments, RFID tag 500 may be attached to a medical device. For example, RFID tag 500 may be attached to a syringe, an IV assembly, a catheter, a container, medication packaging, and/or the like.

In some non-limiting embodiments, RFID tag 500 may be attached to a syringe (e.g., an unfilled syringe, prefilled syringe, needleless syringe, and/or the like). For example, RFID tag 500 may be attached to a syringe cap of the syringe. In such an example, RFID tag 500 may bridge a gap between the syringe cap and the syringe body of the syringe. As another example, RFID tag 500 may be attached to a safety device of the syringe. As another example, RFID tag 500 may be attached to a barrel and to a plunger of the syringe. In such an example, RFID tag 500 may bridge a gap between a barrel and a plunger of the syringe. As another example, RFID tag 500 may be attached to a collar (e.g., a collapsible collar or retractable collar) and/or a connector (e.g., luer connector and/or the like) of the needleless syringe. In such an example, RFID tag 500 may be attached to a collar and/or connector of the needless syringe at one end, and to a mating device (e.g., needle, tubing, luer connector and/or the like) at the opposite end.

In some non-limiting embodiments, RFID tag 500 may be attached to an IV assembly. For example, RFID tag 500 may be positioned between two components of an IV assembly. For example, RFID tag 500 may be positioned between a syringe (e.g., a needleless syringe) and a connector (e.g., an IV connector). In some non-limiting embodiments, RFID tag 500 may be attached to two components of an IV assembly. For example, RFID tag 500 may be attached to a syringe (e.g., a needleless syringe) and/or a connector (e.g., an IV connector).

In some non-limiting embodiments, RFID tag 500 may be attached to a catheter. For example, RFID tag 500 may be attached to a safety device of a catheter. As another example, RFID tag 500 may be attached to a cap (e.g., disinfecting cap) of the catheter. As another example, RFID tag 500 may be attached to a connector (e.g., luer connector and/or the like) of the catheter. In such an example, RFID tag 500 may be attached to a connector of the catheter at one end, and to a mating device (e.g., needleless connector, manifold, catheter line, and/or the like) at the opposite end.

In some non-limiting embodiments, RFID tag 500 may be attached to a container. For example, RFID tag 500 may be attached to a medication container (e.g., medication bottles, medication boxes such as pill boxes, IV bags, and/or the like). In such an example, RFID tag 500 may be attached to a cap of a container (e.g., medication container). For example, RFID tag 500 may bridge a gap between the cap of the container and the body of the container. In some non-limiting embodiments, RFID tag 500 may be attached to a food and/or beverage container. In such an example, RFID tag 500 may be attached to a cap of a food and/or beverage container.

In some non-limiting embodiments, RFID tag 500 may be attached to a packaging for a medical device. In such an example, RFID tag 500 may be attached to a lid and/or a cap of a packaging for a medical device. For example, RFID tag 500 may bridge a gap between the lid and/or cap of a packaging for a medical device and a body of a packaging of a medical device. In some non-limiting embodiments, RFID tag 500 may be attached to medication packaging (e.g., a medication blister pack). In such an example, RFID tag 500 may be provided on the label of the medication packaging.

In some non-limiting embodiments, RFID tag 500 may have a size that allows RFID tag 500 to be attached to a particular medical device. In such an example, the size of RFID tag 500 may be proportional to the size of the medical device or component of the medical device that RFID tag 500 is attached to. As another example, RFID tag 500 may have a size that is sufficient to include each of the components of RFID tag 500 that are to be positioned on RFID tag 500.

In some non-limiting embodiments, RFID tag 500 may have a shape that allows RFID tag 500 to be attached to a particular medical device. In such an example, RFID tag 500 may have a shape that corresponds to an area of a medical device that RFID tag 500 is attached to. As another example, RFID tag 500 may have a shape that is sufficient to include each of the components of RFID tag 500 that are to be positioned on RFID tag 500.

In some non-limiting embodiments, RFID tag 500 may transmit a signal to an RFID reader device (e.g., RFID reader device 104). In some non-limiting embodiments, RFID tag 500 may be a passive RFID tag. In such an example, RFID tag 500 (e.g., RFID circuit 502 of RFID tag 500) may be powered by electromagnetic energy from an electromagnetic wave transmitted by an RFID reader device to interrogate an RFID tag 500. In some non-limiting embodiments, RFID tag 500 may be an active RFID tag. In such an example, RFID tag 500 may be powered by a power source included in RFID tag 500 and, thus, can be read at a greater range by an RFID reader device compared to a passive RFID tag. In some non-limiting embodiments, RFID tag 500 may be read by an RFID reader device at a distance of at least 100 meters. In some non-limiting embodiments, RFID tag 500 may be near-field communication (NFC) tag 500. In some non-limiting embodiments, RFID tag 500 may be an RFID tear tag 500. RFID tear tag 500, as used herein, refers to an RFID tag that includes an electrical component, where the output of RFID tag 500 depends on if the electrical component is in a closed state or an open state.

In some non-limiting embodiments, RFID tag 500 may include RFID circuit 502, a plurality of electrical components 504a, 504b, 504c, and base component 510. In some non-limiting embodiments, RFID circuit 502 may be positioned on base component 510. In such an example, RFID circuit 502 may be attached to base component 510 (e.g., via an adhesive, welding, soldering, and/or the like). In some non-limiting embodiments, a plurality of electrical components 504a, 504b, 504c may be positioned on base component 510. For example, a plurality of electrical components 504a, 504b, 504c may be attached to base component 510 (e.g., via an adhesive, welding, soldering, and/or the like). As another example, a plurality of electrical components 504a, 504b, 504c may be embedded into base component 510. In some non-limiting embodiments, each electrical component of a plurality of electrical components 504a, 504b, 504c may be coupled to RFID circuit 502. For example, each electrical component of a plurality of electrical components 504a, 504b, 504c may be electrically connected to RFID circuit 502.

In some non-limiting embodiments, base component 510 may include attachment point 516 that is configured to allow for base component 510 to be separated into a first section and a plurality of separable sections.

In some non-limiting embodiments, RFID tag 500 may include RFID circuit 502. In some non-limiting embodiments, RFID circuit 502 may be the same as RFID circuit 402. In some non-non-limiting embodiments, RFID circuit 502 may provide a signal based on a state of one or more components of RFID tag 500. For example, RFID circuit 502 may provide a certain signal when one or more components of RFID tag 500 are in a first state, and provide a different signal when one or more components of RFID tag 500 are in a second state.

In some non-limiting embodiments, a plurality of electrical components 504a, 504b, 504c may include first electrical component 504a. In some non-limiting embodiments, first electrical component 504a may be the same as electrical component 404. In some non-limiting embodiments, first electrical component 504a may include first portion 506a and second portion 508a. In some non-limiting embodiments, first portion 506a and second portion 508a of first electrical component 504a may be the same as first portion 406 and second portion 408 of electrical component 404.

In some non-limiting embodiments, a plurality of electrical components 504a, 504b, 504c may include second electrical component 504b. In some non-limiting embodiments, second electrical component 504b may be the same as electrical component 404. In some non-limiting embodiments, second electrical component 504b may include first portion 506b and second portion 508b. In some non-limiting embodiments, first portion 506b and second portion 508b of second electrical component 504b may be the same as first portion 406 and second portion 408 of electrical component 404.

In some non-limiting embodiments, a plurality of electrical components 504a, 504b, 504c may include third electrical component 504c. In some non-limiting embodiments, third electrical component 504c may be the same as electrical component 404. In some non-limiting embodiments, third electrical component 504c may include first portion 506c and second portion 508c.

In some non-limiting embodiments, first portion 506c and second portion 508c of third electrical component 504c may be the same as first portion 406 and second portion 408 of electrical component 404.

It can be readily envisioned that additional electrical components, such as a fourth electrical component, fifth electrical component, and/or the like, with the same characteristics as a plurality of electrical components 504a, 504b, 504c may be included in RFID tag 500. For example, RFID tag 500 may include at least two, or at least three, or at least four, or at least five electrical components 504, where each electrical component 504 may include an accompanying separable portion (e.g., one of separable portions 514a, 514b, or 514c) of base component 510.

In some non-limiting embodiments, base component 510 may be the same as base component 410. In some non-limiting embodiments, base component 510 may include first portion 512 and a plurality of separable portions 514a, 514b, 514c. In some non-limiting embodiments, first portion 512 of base component 510 may be the same as first portion 412 of base component 410.

In some non-limiting embodiments, a plurality of separable portions 514a, 514b, 514c may include first separable portion 514a. In some non-limiting embodiments, second portion 508a of first electrical component 504a may be positioned on first separable portion 514a of base component 510. For example, second portion 508a of first electrical component 504a may be attached to first separable portion 514a of base component 510 (e.g., via an adhesive, welding, soldering, and/or the like). As another example, second portion 508a of first electrical component 504a may be embedded into first separable portion 514a of base component 510.

In some non-limiting embodiments, a plurality of separable portions 514a, 514b, 514c may include second separable portion 514b. In some non-limiting embodiments, second portion 508b of second electrical component 504b may be positioned on second separable portion 514b of base component 510. For example, second portion 508b of second electrical component 504b may be attached to second separable portion 514b of base component 510 (e.g., via an adhesive, welding, soldering, and/or the like). As another example, second portion 508b of second electrical component 504b may be embedded into second separable portion 514b of base component 510.

In some non-limiting embodiments, a plurality of separable portions 514a, 514b, 514c may include third separable portion 514c. In some non-limiting embodiments, second portion 508c of third electrical component 504c may be positioned on third separable portion 514c of base component 510. For example, second portion 508c of third electrical component 504c may be attached to third separable portion 514c of base component 510 (e.g., via an adhesive, welding, soldering, and/or the like). As another example, second portion 508c of third electrical component 504c may be embedded into third separable portion 514c of base component 510.

In some non-limiting embodiments, base component 510 may include attachment point 516 that separates first portion 512 of base component 510 from a plurality of separable portions 514a, 514b, 514c of base component 510. In some non-limiting embodiments, attachment point 516 may be the same as attachment point 416.

In some non-limiting embodiments, first portion 512 and a plurality of separable portions 514a, 514b, 514c of base component 510 may form continuous, unitary base component 510 at attachment point 516. In such an example, first portion 512 of base component 510 is materially connected to each separable portion of a plurality of separable portions 514a, 514b, 514c of base component 510 at attachment point 516. In some non-limiting embodiments, first portion 512 of base component 510 may be separate from each separable portion of a plurality of separable portions 514a, 514b, 514c of base component 510 at attachment point 516, such that first portion 512 and a plurality of separable portions 514a, 514b, 514c of base component 510 are not materially connected, but are coupled at attachment point 516.

In some non-limiting embodiments, first portion 506a and second portion 508a of first electrical component 504a may be coupled together when first electrical component 504a is in a first state. For example, first portion 506a and second portion 508a of first electrical component 504a may be electrically coupled (e.g., conductively coupled) together when first electrical component 504a is in a first state. In some non-limiting embodiments, first portion 512 and first separable portion 514a of base component 510 may be coupled together, such that first portion 506a and second portion 508a of first electrical component 504a are coupled together, when first electrical component 504a is in a first state.

In some non-limiting embodiments, first portion 506b and second portion 508b of second electrical component 504b may be coupled together when second electrical component 504b is in a first state. For example, first portion 506b and second portion 508b of second electrical component 504b may be electrically coupled (e.g., conductively coupled) together when second electrical component 504b is in a first state. In some non-limiting embodiments, first portion 512 and second separable portion 514b of base component 510 may be coupled together, such that first portion 506b and second portion 508b of second electrical component 504b are coupled together, when second electrical component 504b is in a first state.

In some non-limiting embodiments, first portion 506c and second portion 508c of third electrical component 504c may be coupled together when third electrical component 504c is in a first state. For example, first portion 506c and second portion 508c of third electrical component 504c may be electrically coupled (e.g., conductively coupled) together when third electrical component 504c is in a first state. In some non-limiting embodiments, first portion 512 and third separable portion 514c of base component 510 may be coupled together, such that first portion 506c and second portion 508c of third electrical component 504c are coupled together, when third electrical component 504c is in a first state.

In some non-limiting embodiments, RFID circuit 502 may be configured to provide a first signal, such as during interrogation by an RFID reader device, when first electrical component 504a, second electrical component 504b, and/or third electrical component 504c is in a first state. In some non-limiting embodiments, a first signal provided by RFID circuit 502 may include data associated with an indication of a first state of a medical device. In some non-limiting embodiments, data associated with a first state of a medical device may indicate a closed configuration. For example, data associated with a first state of a medical device may indicate a closed configuration for the medical device. In such an example, data associated with a first state of a medical device may indicate that a cap and/or lid of a medical device (e.g. container and/or the like) is secured to a body of the medical device to seal the contents (e.g., medication) within the medical device.

In some non-limiting embodiments, data associated with a first state of a medical device may indicate an unused configuration. For example, data associated with a first state of a medical device may indicate an unused configuration for the medical device. For example, data associated with a first state of a medical device may indicate that a medication has not been delivered by the medical device (e.g., syringe and/or the like). In another example, data associated with a first state of a medical device may indicate that a safety device of the medical device (e.g., syringe, catheter, and/or the like) has not been activated. In another example, data associated with a first state of a medical device may indicate that a collar (e.g., collapsible collar, retractable collar, and/or the like) of the medical device has not been displaced in order to connect the medical device to a mating device.

In some non-limiting embodiments, data associated with a first state of a medical device may indicate a coupled configuration. For example, data associated with a first state of a medical device may indicate a coupled configuration for a medical device. For example, data associated with a first state of a medical device may indicate that a first component of a medical device is coupled to a second component of a medical device. In such an example, data associated with a first state of a medical device may indicate that a first connector (e.g., a luer connector and/or the like) is connected to a second connector (e.g., a luer connector and/or the like), such as, for example, the connection between a needleless syringe and an IV connector.

In some non-limiting embodiments, data associated with a first state of a medical device may indicate an uncontaminated configuration. For example, data associated with a first state of a medical device may indicate an uncontaminated configuration of a medical device. For example, data associated with a first state of a medical device may indicate that a floating collar of the medical device has not been bumped or touched prior to connection (e.g., luer attachment and/or the like). In another example, data associated with a first state of a medical device may indicate that a deformable element in a collar of a medical device (e.g., a syringe and/or the like) has not been deformed.

In some non-limiting embodiments, a first signal provided by RFID circuit 502 may include data associated with an indication of a first state of a packaging of a medical device. In some non-limiting embodiments, data associated with a first state of a packaging of a medical device may indicate a sealed configuration. For example, data associated with a first state of a packaging of a medical device may indicate a sealed configuration of a packaging of a medical device. In such an example, data associated with a first state of a packaging of a medical device may indicate that the packaging has yet to be open to remove the medical device from the packaging. In another example, data associated with a first state of a packaging of a medical device may indicate that a seal holding a medication in the packaging has not been broken (e.g., a medication blister pack and/or the like).

In some non-limiting embodiments, first portion 506a and second portion 508a of first electrical component 504a may be decoupled when first electrical component 504a is in a second state. For example, first portion 506a and second portion 508a of first electrical component 504a may not be electrically coupled (e.g., not conductively coupled) when first electrical component 504a is in a second state. In some non-limiting embodiments, first portion 512 of base component 510 may be decoupled from first separable portion 514a of base component 510 at attachment point 516, such that first portion 506a and second portion 508a of first electrical component 504a are decoupled, when first electrical component 504a is in a second state. For example, first portion 512 of base component 510 may be decoupled from first separable portion 514a of base component 510 at attachment point 516, such that there is a gap between first portion 512 and first separable portion 514a of base component 510, where the gap is sufficiently wide, such that first portion 506a and second portion 508a of first electrical component 504a are not electrically coupled. In some non-limiting embodiments, first portion 512 of base component 510 may be decoupled from first separable portion 514a of base component 510 by pulling and/or rotating first portion 512 of base component 510 away from first separable portion 514a of base component 510 at attachment point 516, such as to form a gap with a width between first portion 512 and first separable portion 514a of base component 510. For example, base component 510 may be broke at attachment point 516, and then first portion 512 of base component 510 may be pulled and/or rotated away from first separable portion 514a of base component 510.

In some non-limiting embodiments, first portion 506b and second portion 508b of second electrical component 504b may be decoupled when second electrical component 504b is in a second state. For example, first portion 506b and second portion 508b of second electrical component 504b may not be electrically coupled (e.g., not conductively coupled) when second electrical component 504b is in a second state. In some non-limiting embodiments, first portion 512 of base component 510 may be decoupled from second separable portion 514b of base component 510 at attachment point 516, such that first portion 506b and second portion 508b of second electrical component 504b are decoupled, when second electrical component 504b is in a second state. For example, first portion 512 of base component 510 may be decoupled from second separable portion 514b of base component 510 at attachment point 516, such that there is a gap between first portion 512 and second separable portion 514b of base component 510, where the gap is sufficiently wide, such that first portion 506b and second portion 508b of second electrical component 504b are not electrically coupled. In some non-limiting embodiments, first portion 512 of base component 510 may be decoupled from second separable portion 514b of base component 510 by pulling and/or rotating first portion 512 of base component 510 away from second separable portion 514b of base component 510 at attachment point 516, such as to form a gap with a width between first portion 512 and second separable portion 514b of base component 510. For example, base component 510 may be broke at attachment point 516, and then first portion 512 of base component 510 may be pulled and/or rotated away from second separable portion 514b of base component 510.

In some non-limiting embodiments, first portion 506c and second portion 508c of third electrical component 504c may be decoupled when third electrical component 504c is in a second state. For example, first portion 506c and second portion 508c of third electrical component 504c may not be electrically coupled (e.g., not conductively coupled) when third electrical component 504c is in a second state. In some non-limiting embodiments, first portion 512 of base component 510 may be decoupled from third separable portion 514c of base component 510 at attachment point 516, such that first portion 506c and second portion 508c of third electrical component 504c are decoupled, when third electrical component 504c is in a second state. For example, first portion 512 of base component 510 may be decoupled from third separable portion 514c of base component 510 at attachment point 516 such that there is a gap between first portion 512 and third separable portion 514c of base component 510, where the gap is sufficiently wide such that first portion 506c and second portion 508c of third electrical component 504c are not electrically coupled. In some non-limiting embodiments, first portion 512 of base component 510 may be decoupled from third separable portion 514c of base component 510 by pulling and/or rotating first portion 512 of base component 510 away from third separable portion 514c of base component 510 at attachment point 516, such as to form a gap with a width between first portion 512 and third separable portion 514c of base component 510. For example, base component 510 may be broke at attachment point 516, and then first portion 512 of base component 510 may be pulled and/or rotated away from third separable portion 514c of base component 510.

In some non-limiting embodiments, RFID circuit 502 may be configured to provide a second signal, such as during interrogation by an RFID reader device, when first electrical component 504a, second electrical component 504b, and/or third electrical component 504c is in a second state. In some non-limiting embodiments, the second signal provided by RFID circuit 502 may be different from the first signal provided by RFID circuit 502.

In some non-limiting embodiments, a second signal provided by RFID circuit 502 may include data associated with an indication of a second state of a medical device. In some non-limiting embodiments, data associated with a second state of a medical device may indicate an open configuration. For example, data associated with a second state of a medical device may indicate an open configuration for the medical device. In such an example, data associated with a second state of a medical device may indicate that a cap and/or lid of a medical device (e.g. container and/or the like) has been removed from a body of the medical device, such that contents (e.g., medication) within the medical device are no longer sealed within the medical device.

In some non-limiting embodiments, data associated with a second state of a medical device may indicate an unfilled configuration. For example, data associated with a second state of a medical device may indicate an unfilled configuration for the medical device. For example, data associated with a second state of a medical device may indicate that a medical device (e.g., syringe and/or the like) has yet to receive a medication.

In some non-limiting embodiments, data associated with a second state of a medical device may indicate a used configuration. For example, data associated with a second state of a medical device may indicate a used configuration for the medical device. For example, data associated with a second state of a medical device may indicate that a medication has been delivered by the medical device (e.g., syringe and/or the like). In another example, data associated with a second state of a medical device may indicate that a safety device of the medical device (e.g., syringe, catheter, and/or the like) has been activated. In another example, data associated with a second state of a medical device may indicate that a collar (e.g., collapsible collar, retractable collar, and/or the like) of the medical device has been displaced in order to connect the medical device to a mating device.

In some non-limiting embodiments, data associated with a second state of a medical device may indicate a decoupled configuration. For example, data associated with a second state of a medical device may indicate a decoupled configuration for a medical device. For example, data associated with a second state of a medical device may indicate that a first component of a medical device is not coupled to a second component of a medical device. In such an example, data associated with a second state of a medical device may indicate that a first connector (e.g., a luer connector and/or the like) is not connected to a second connector (e.g., a luer connector and/or the like), such as, for example, the connection between a needleless syringe and an IV connector.

In some non-limiting embodiments, data associated with a second state of a medical device may indicate a contaminated configuration. For example, data associated with a second state of a medical device may indicate a contaminated configuration of a medical device. For example, data associated with a second state of a medical device may indicate that a floating collar of the medical device has been bumped or touched prior to connection (e.g., luer attachment and/or the like). In another example, data associated with a second state of a medical device may indicate that a deformable element in a collar of a medical device (e.g., a syringe and/or the like) has been deformed.

In some non-limiting embodiments, a second signal provided by RFID circuit 502 may include data associated with an indication of a second state of a packaging of a medical device. In some non-limiting embodiments, data associated with a second state of a packaging of a medical device may indicate an unsealed configuration. For example, data associated with a second state of a packaging of a medical device may indicate an unsealed configuration of a packaging of a medical device. In such an example, data associated with a second state of a packaging of a medical device may indicate that the packaging has been opened to remove the medical device from the packaging. In another example, data associated with a second state of a packaging of a medical device may indicate that a seal holding a medication in the packaging has been broken (e.g., a medication blister pack and/or the like).

In some non-limiting embodiments, first portion 506b and second portion 508b of second electrical component 504b may be coupled together when second electrical component 504b is in a third state. For example, first portion 506b and second portion 508b of second electrical component 504b may be electrically coupled (e.g., conductively coupled) together when second electrical component 504b is in a third state. In some non-limiting embodiments, first portion 512 and second separable portion 514b of base component 510 may be coupled together, such that first portion 506b and second portion 508b of second electrical component 504b are coupled together, when second electrical component 504b is in a third state.

In some non-limiting embodiments, RFID circuit 502 may be configured to provide a third signal, such as during interrogation by an RFID reader device, when second electrical component 504b is in a third state. In some non-limiting embodiments, the third signal provided by RFID circuit 502 may be different from both the first signal and the second signal provided by RFID circuit 502.

In some non-limiting embodiments, a third signal provided by RFID circuit 502 may include data associated with an indication of a third state of a medical device. In some non-limiting embodiments, data associated with a third state of a medical device may indicate a closed configuration. For example, data associated with a third state of a medical device may indicate a closed configuration for the medical device. In such an example, data associated with a third state of a medical device may indicate a cap and/or lid of a medical device (e.g. container and/or the like) is secured to a body of the medical device to seal the contents (e.g., medication) within the medical device.

In some non-limiting embodiments, data associated with a third state of a medical device may indicate an unused configuration. For example, data associated with a third state of a medical device may indicate an unused configuration for the medical device. For example, data associated with a third state of a medical device may indicate that a medication has not been delivered by the medical device (e.g., syringe and/or the like). In another example, data associated with a third state of a medical device may indicate that a safety device of the medical device (e.g., syringe, catheter, and/or the like) has not been activated. In another example, data associated with a third state of a medical device may indicate that a collar (e.g., collapsible collar, retractable collar, and/or the like) of the medical device has not been displaced in order to connect the medical device to a mating device.

In some non-limiting embodiments, data associated with a third state of a medical device may indicate a coupled configuration. For example, data associated with a third state of a medical device may indicate a coupled configuration for a medical device. For example, data associated with a third state of a medical device may indicate that a first component of a medical device is coupled to a second component of a medical device. In such an example, data associated with a third state of a medical device may indicate that a first connector (e.g., a luer connector and/or the like) is connected to a second connector (e.g., a luer connector and/or the like), such as, for example, the connection between a needleless syringe and an IV connector.

In some non-limiting embodiments, data associated with a third state of a medical device may indicate an uncontaminated configuration. For example, data associated with a third state of a medical device may indicate an uncontaminated configuration of a medical device. For example, data associated with a third state of a medical device may indicate that a floating collar of the medical device has not been bumped or touched prior to connection (e.g., luer attachment and/or the like). In another example, data associated with a third state of a medical device may indicate that a deformable element in a collar of a medical device (e.g., a syringe and/or the like) has not been deformed.

In some non-limiting embodiments, a third signal provided by RFID circuit 502 may include data associated with an indication of a third state of a packaging of a medical device. In some non-limiting embodiments, data associated with a third state of a packaging of a medical device may indicate a sealed configuration. For example, data associated with a third state of a packaging of a medical device may indicate a sealed configuration of a packaging of a medical device. In such an example, data associated with a third state of a packaging of a medical device may indicate that the packaging has yet to be open to remove the medical device from the packaging. In another example, data associated with a third state of a packaging of a medical device may indicate that a seal holding a medication in the packaging has not been broken (e.g., a medication blister pack and/or the like).

In some non-limiting embodiments, first portion 506b and second portion 508b of second electrical component 504b may be decoupled when second electrical component 504b is in a fourth state. For example, first portion 506b and second portion 508b of second electrical component 504b may not be electrically coupled (e.g., not conductively coupled) when second electrical component 504b is in a fourth state. In some non-limiting embodiments, first portion 512 of base component 510 may be decoupled from second separable portion 514b of base component 510 at attachment point 516, such that first portion 506b and second portion 508b of second electrical component 504b are decoupled, when second electrical component 504b is in a fourth state. For example, first portion 512 of base component 510 may be decoupled from second separable portion 514b of base component 510 at attachment point 516, such that there is a gap between first portion 512 and second separable portion 514b of base component 510, where the gap is sufficiently wide, such that first portion 506b and second portion 508b of second electrical component 504b are not electrically coupled.

In some non-limiting embodiments, RFID circuit 502 may be configured to provide a fourth signal, such as during interrogation by an RFID reader device, when second electrical component 504b is in a fourth state. In some non-limiting embodiments, the fourth signal provided by RFID circuit 502 may be different from the first signal, the second signal, and the third signal provided by RFID circuit 502.

In some non-limiting embodiments, a fourth signal provided by RFID circuit 502 may include data associated with an indication of a fourth state of a medical device. In some non-limiting embodiments, data associated with a fourth state of a medical device may indicate an open configuration. For example, data associated with a fourth state of a medical device may indicate an open configuration for the medical device. In such an example, data associated with a fourth state of a medical device may indicate that a cap and/or lid of a medical device (e.g. container and/or the like) has been removed from a body of the medical device, such that contents (e.g., medication) within the medical device are no longer sealed within the medical device.

In some non-limiting embodiments, data associated with a fourth state of a medical device may indicate an unfilled configuration. For example, data associated with a fourth state of a medical device may indicate an unfilled configuration for the medical device. For example, data associated with a fourth state of a medical device may indicate that a medical device (e.g., syringe and/or the like) has yet to receive a medication.

In some non-limiting embodiments, data associated with a fourth state of a medical device may indicate a used configuration. For example, data associated with a fourth state of a medical device may indicate a used configuration for the medical device. For example, data associated with a fourth state of a medical device may indicate that a medication has been delivered by the medical device (e.g., syringe and/or the like). In another example, data associated with a fourth state of a medical device may indicate that a safety device of the medical device (e.g., syringe, catheter, and/or the like) has been activated. In another example, data associated with a fourth state of a medical device may indicate that a collar (e.g., collapsible collar, retractable collar, and/or the like) of the medical device has been displaced in order to connect the medical device to a mating device.

In some non-limiting embodiments, data associated with a fourth state of a medical device may indicate a decoupled configuration. For example, data associated with a fourth state of a medical device may indicate a decoupled configuration for a medical device. For example, data associated with a fourth state of a medical device may indicate that a first component of a medical device is not coupled to a second component of a medical device. In such an example, data associated with a fourth state of a medical device may indicate that a first connector (e.g., a luer connector and/or the like) is not connected to a second connector (e.g., a luer connector and/or the like), such as, for example, the connection between a needleless syringe and an IV connector.

In some non-limiting embodiments, data associated with a fourth state of a medical device may indicate a contaminated configuration. For example, data associated with a fourth state of a medical device may indicate a contaminated configuration of a medical device. For example, data associated with a fourth state of a medical device may indicate that a floating collar of the medical device has been bumped or touched prior to connection (e.g., luer attachment and/or the like). In another example, data associated with a fourth state of a medical device may indicate that a deformable element in a collar of a medical device (e.g., a syringe and/or the like) has been deformed.

In some non-limiting embodiments, a fourth signal provided by RFID circuit 502 may include data associated with an indication of a fourth state of a packaging of a medical device. In some non-limiting embodiments, data associated with a fourth state of a packaging of a medical device may indicate an unsealed configuration. For example, data associated with a fourth state of a packaging of a medical device may indicate an unsealed configuration of a packaging of a medical device. In such an example, data associated with a fourth state of a packaging of a medical device may indicate that the packaging has been opened to remove the medical device from the packaging. In another example, data associated with a fourth state of a packaging of a medical device may indicate that a seal holding a medication in the packaging has been broken (e.g., a medication blister pack and/or the like).

In some non-limiting embodiments, first portion 506c and second portion 508c of third electrical component 504c may be coupled together when third electrical component 504c is in a fifth state. For example, first portion 506c and second portion 508c of third electrical component 504c may be electrically coupled (e.g., conductively coupled) together when third electrical component 504c is in a fifth state. In some non-limiting embodiments, first portion 512 and third separable portion 514c of base component 510 may be coupled together, such that first portion 506c and second portion 508c of third electrical component 504c are coupled together, when third electrical component 504c is in a fifth state.

In some non-limiting embodiments, RFID circuit 502 may be configured to provide a fifth signal, such as during interrogation by an RFID reader device, when third electrical component 504c is in a fifth state. In some non-limiting embodiments, the fifth signal provided by RFID circuit 502 may be different from the first signal, the second signal, the third signal, and the fourth signal provided by RFID circuit 502.

In some non-limiting embodiments, a fifth signal provided by RFID circuit 502 may include data associated with an indication of a fifth state of a medical device. In some non-limiting embodiments, data associated with a fifth state of a medical device may indicate a closed configuration. For example, data associated with a fifth state of a medical device may indicate a closed configuration for the medical device. In such an example, data associated with a fifth state of a medical device may indicate that a cap and/or lid of a medical device (e.g. container and/or the like) is secured to a body of the medical device to seal the contents (e.g., medication) within the medical device.

In some non-limiting embodiments, data associated with a fifth state of a medical device may indicate an unused configuration. For example, data associated with a fifth state of a medical device may indicate an unused configuration for the medical device. For example, data associated with a fifth state of a medical device may indicate that a medication has not been delivered by the medical device (e.g., syringe and/or the like). In another example, data associated with a fifth state of a medical device may indicate that a safety device of the medical device (e.g., syringe, catheter, and/or the like) has not been activated. In another example, data associated with a fifth state of a medical device may indicate that a collar (e.g., collapsible collar, retractable collar, and/or the like) of the medical device has not been displaced in order to connect the medical device to a mating device.

In some non-limiting embodiments, data associated with a fifth state of a medical device may indicate a coupled configuration. For example, data associated with a fifth state of a medical device may indicate a coupled configuration for a medical device. For example, data associated with a fifth state of a medical device may indicate that a first component of a medical device is coupled to a second component of a medical device. In such an example, data associated with a fifth state of a medical device may indicate that a first connector (e.g., a luer connector and/or the like) is connected to a second connector, such as, for example, the connection between a needleless syringe and an IV connector.

In some non-limiting embodiments, data associated with a fifth state of a medical device may indicate an uncontaminated configuration. For example, data associated with a fifth state of a medical device may indicate an uncontaminated configuration of a medical device. For example, data associated with a fifth state of a medical device may indicate that a floating collar of the medical device has not been bumped or touched prior to connection (e.g., luer attachment and/or the like). In another example, data associated with a fifth state of a medical device may indicate that a deformable element in a collar of a medical device (e.g., a syringe and/or the like) has not been deformed.

In some non-limiting embodiments, a fifth signal provided by RFID circuit 502 may include data associated with an indication of a fifth state of a packaging of a medical device. In some non-limiting embodiments, data associated with a fifth state of a packaging of a medical device may indicate a sealed configuration. For example, data associated with a fifth state of a packaging of a medical device may indicate a sealed configuration of a packaging of a medical device. In such an example, data associated with a fifth state of a packaging of a medical device may indicate that the packaging has yet to be open to remove the medical device from the packaging. In another example, data associated with a fifth state of a packaging of a medical device may indicate that a seal holding a medication in the packaging has not been broken (e.g., a medication blister pack and/or the like).

In some non-limiting embodiments, first portion 506c and second portion 508c of third electrical component 504c may be decoupled when third electrical component 504c is in a sixth state. For example, first portion 506c and second portion 508c of third electrical component 504c may not be electrically coupled (e.g., not conductively coupled) when third electrical component 504c is in a sixth state. In some non-limiting embodiments, first portion 512 of base component 510 may be decoupled from third separable portion 514c of base component 510 at attachment point 516, such that first portion 506c and second portion 508c of third electrical component 504c are decoupled, when third electrical component 504c is in a sixth state. For example, first portion 512 of base component 510 may be decoupled from third separable portion 514c of base component 510 at attachment point 516, such that there is a gap between first portion 512 and third separable portion 514c of base component 510, where the gap is sufficiently wide, such that first portion 506c and second portion 508c of third electrical component 504c are not electrically coupled.

In some non-limiting embodiments, RFID circuit 502 may be configured to provide a sixth signal, such as during interrogation by an RFID reader device, when third electrical component 504c is in a sixth state. In some non-limiting embodiments, the sixth signal provided by RFID circuit 502 may be different from the first signal, the second signal, the third signal, the fourth signal, and the fifth signal provided by RFID circuit 502.

In some non-limiting embodiments, a sixth signal provided by RFID circuit 502 may include data associated with an indication of a sixth state of a medical device. In some non-limiting embodiments, data associated with a sixth state of a medical device may indicate an open configuration. For example, data associated with a sixth state of a medical device may indicate an open configuration for the medical device. In such an example, data associated with a sixth state of a medical device may indicate that a cap and/or lid of a medical device (e.g. container and/or the like) has been removed from a body of the medical device such that contents (e.g., medication) within the medical device are no longer sealed within the medical device.

In some non-limiting embodiments, data associated with a sixth state of a medical device may indicate an unfilled configuration. For example, data associated with a sixth state of a medical device may indicate an unfilled configuration for the medical device. For example, data associated with a sixth state of a medical device may indicate that a medical device (e.g., syringe and/or the like) has yet to receive a medication.

In some non-limiting embodiments, data associated with a sixth state of a medical device may indicate a used configuration. For example, data associated with a sixth state of a medical device may indicate a used configuration for the medical device. For example, data associated with a sixth state of a medical device may indicate that a medication has been delivered by the medical device (e.g., syringe and/or the like). In another example, data associated with a sixth state of a medical device may indicate that a safety device of the medical device (e.g., syringe, catheter, and/or the like) has been activated. In another example, data associated with a sixth state of a medical device may indicate that a collar (e.g., collapsible collar, retractable collar, and/or the like) of the medical device has been displaced in order to connect the medical device to a mating device.

In some non-limiting embodiments, data associated with a sixth state of a medical device may indicate a decoupled configuration. For example, data associated with a sixth state of a medical device may indicate a decoupled configuration for a medical device. For example, data associated with a sixth state of a medical device may indicate that a first component of a medical device not coupled to a second component of a medical device. In such an example, data associated with a sixth state of a medical device may indicate that a first connector (e.g., a luer connector and/or the like) is not connected (e.g., secured) to a second connector, such as, for example, the connection between a needleless syringe and an IV connector.

In some non-limiting embodiments, data associated with a sixth state of a medical device may indicate a contaminated configuration. For example, data associated with a sixth state of a medical device may indicate a contaminated configuration of a medical device. For example, data associated with a sixth state of a medical device may indicate that a floating collar of the medical device has been bumped or touched prior to connection (e.g., luer attachment and/or the like). In another example, data associated with a sixth state of a medical device may indicate that a deformable element in a collar of a medical device (e.g., a syringe and/or the like) has been deformed.

In some non-limiting embodiments, a sixth signal provided by RFID circuit 502 may include data associated with an indication of a sixth state of a packaging of a medical device. In some non-limiting embodiments, data associated with a sixth state of a packaging of a medical device may indicate an unsealed configuration. For example, data associated with a sixth state of a packaging of a medical device may indicate an unsealed configuration of a packaging of a medical device. In such an example, data associated with a sixth state of a packaging of a medical device may indicate that the packaging has been opened to remove the medical device from the packaging. In another example, data associated with a sixth state of a packaging of a medical device may indicate that a seal holding a medication in the packaging has been broken (e.g., a medication blister pack and/or the like).

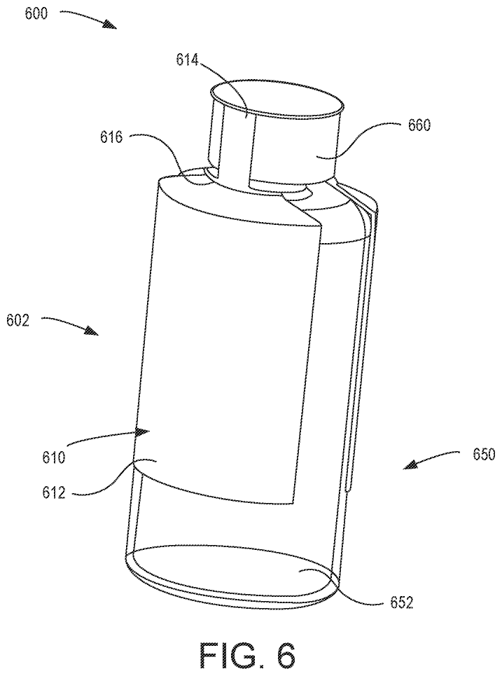

Referring to FIG. 6, FIG. 6 is a diagram of medical device assembly 600. As shown in FIG. 6, medical device assembly 600 includes RFID tag 602 positioned on container 650. In some non-limiting embodiments, RFID tag 602 may be attached to container 650 (e.g., via an adhesive and/or the like). In some non-limiting embodiments, RFID tag 602 may be the same as RFID tag 400 and/or RFID tag 500.

In some non-limiting embodiments, container 650 may have a size that is appropriate based on the contents to be contained within container 650 and where container 650 is to be located. In some non-limiting embodiments, container 650 may have a shape that is appropriate based on the contents to be contained within container 650 and where container 650 is to be located. For example, the cross-section of container 650 may be a rectangle, a square, a circle, an oval, and/or the like. In some non-limiting embodiments, container 650 may include a material that is appropriate based on the contents to be contained within container 650. For example, the material of container 650 may include plastic, glass, and/or the like. In some non-limiting embodiments, container 650 may be configured to contain contents within container 650. For example, container 650 may be configured to contain solids and/or liquids within container 650. In such an example, container 650 may be configured to contain medication within container 650.

In some non-limiting embodiments, container 650 may include container body 652. In some non-limiting embodiments, container body 652 may have a size that is appropriate based on the contents to be contained within container body 652 and where container 650 is to be located. In some non-limiting embodiments, container body 652 may have a shape that is appropriate based on the contents to be contained within container body 652 and where container 650 is to be located. For example, the cross-section of container body 652 may be a rectangle, a square, a circle, an oval, and/or the like. In some non-limiting embodiments, container body 652 include a material that is appropriate based on the contents to be contained within container body 652. For example, the material of container body 652 may include plastic, glass, and/or the like.

In some non-limiting embodiments, container 650 may include closure 660. In some non-limiting embodiments, closure 660 may include a cap, a top, a seal, and/or the like. In some non-limiting embodiments, closure 660 may have a size that is appropriate to be coupled (e.g., secured) to container body 652. For example, closure 660 may have a size that is proportional to the size of container body 652. In some non-limiting embodiments, closure 660 may have a shape that is appropriate to be secured to container body 652. For example, a shape of the cross-section of closure 660 may be a rectangle, a square, a circle, an oval, and/or the like. In some non-limiting embodiments, closure 660 may include a material that is appropriate based on an application which container 650 is designed. For example, the material of closure 660 may include plastic, glass, and/or the like.

In some non-limiting embodiments, closure 660 may be positioned over an opening of container body 652. For example, closure 660 may be coupled to (e.g., attached to; seal, and/or the like) an opening of container body 652.

In some non-limiting embodiments, RFID tag 602 may be attached to container 650. In some non-limiting embodiments, RFID tag 602 may have a size that allows RFID tag 602 to be attached to container 650. In such an example, the size of RFID tag 602 may be proportional to the size of container 650. As another example, RFID tag 602 may have a size that is sufficient to include each of the components of RFID tag 602 that are to be positioned on RFID tag 602.

In some non-limiting embodiments, RFID tag 602 may have a shape that allows RFID tag 602 to be attached to container 650. In such an example, RFID tag 602 may have a shape that corresponds to container body 652 and/or to closure 660.

In some non-limiting embodiments, RFID tag 602 may include an RFID circuit, an electrical component, and base component 610. In some non-limiting embodiments, base component 610 may include attachment point 616 that is configured to allow for base component 610 to be separated into a first section and a second section.

In some non-limiting embodiments, RFID tag 602 may include the RFID circuit. In some non-limiting embodiments, the RFID circuit may be the same as RFID circuit 402. In some non-limiting embodiments, the RFID circuit may provide a signal based on a state of one or more components of RFID tag 602. For example, the RFID circuit may provide a first signal when one or more components of RFID tag 602 are in a first state, and provide a second signal when one or more components of RFID tag 602 are in a second state.

In some non-limiting embodiments, RFID tag 602 may include an electrical component. In some non-limiting embodiments, the electrical component may be the same as electrical component 404. In some non-limiting embodiments, the electrical component may include a first portion and a second portion. In some non-limiting embodiments, the first portion and the second portion of the electrical component may be the same as first portion 406 and second portion 408 of electrical component 404.

In some non-limiting embodiments, base component 610 may be the same as base component 410. In some non-limiting embodiments, base component 610 may be positioned on container 650. In some non-limiting embodiments, base component 610 may be attached to (e.g., via an adhesive and/or the like) container 650. For example, base component 610 may be attached to container body 652 and/or closure 660 of container 650.

In some non-limiting embodiments, base component 610 may include first portion 612 and second portion 614. In some non-limiting embodiments, first portion 612 and second portion 614 of base component 610 may be the same as first portion 412 and second portion 414 of base component 410. In some non-limiting embodiments, first portion 612 of base component 610 may be positioned on container body 652. For example, first portion 612 of base component 610 may be attached to (e.g., via an adhesive and/or the like) container body 652. In some non-limiting embodiments, first portion 612 of base component 610 may be positioned on closure 660. For example, first portion 612 of base component 610 may be attached to (e.g., via an adhesive and/or the like) closure 660.

In some non-limiting embodiments, second portion 614 of base component 610 may be positioned on container body 652. For example, second portion 614 of base component 610 may be attached to (e.g., via an adhesive and/or the like) container body 652. In some non-limiting embodiments, second portion 614 of base component 610 may be positioned on closure 660. For example, second portion 614 of base component 610 may be attached to (e.g., via an adhesive and/or the like) closure 660.

In some non-limiting embodiments, base component 610 may include attachment point 616 that separates first portion 612 of base component 610 from second portion 614 of base component 610. In some non-limiting embodiments, attachment point 616 may be the same as attachment point 416. In some non-limiting embodiments, attachment point 616 may be positioned at a gap between closure 660 and container body 652. For example, attachment point 616 may bridge the gap between closure 660 and container body 652.

In some non-limiting embodiments, the first portion and the second portion of the electrical component may be coupled together when the electrical component is in a first state. For example, the first portion and the second portion of the electrical component may be electrically coupled (e.g., conductively coupled) together when the electrical component is in a first state. In some non-limiting embodiments, first portion 612 and second portion 614 of base component 610 may be coupled together, such that the first portion and the second portion of the electrical component are coupled together, when the electrical component is in a first state. For example, closure 660 may be coupled to (e.g., attached to and/or the like) container body 652, such that first portion 612 and second portion 614 of base component 610 may be coupled together and the first portion and the second portion of the electrical component are coupled together when the electrical component is in a first state.

In some non-limiting embodiments, the first portion and the second portion of the electrical component may be recoupled after previously being decoupled when the electrical component is in the first state. For example, the first portion and the second portion of the electrical component may be electrically recoupled (e.g., conductively recoupled) after previously being electrically decoupled (e.g., conductively decoupled) when the electrical component is in a first state. In some non-limiting embodiments, first portion 612 and second portion 614 of base component 610 may be recoupled after previously being decoupled, such that the first portion and the second portion of the electrical component are recoupled after previously being decoupled, when the electrical component is in a first state. For example, closure 660 may be recoupled to (e.g., reattached to and/or the like) container body 652 after previously being removed from container body 652, such that first portion 612 and second portion 614 of base component 610 may be recoupled together, and the first portion and the second portion of the electrical component are recoupled together when the electrical component is in a first state. In some non-limiting embodiments, the first portion and the second portion of the electrical component may be decoupled and then recoupled to change the state of the electrical component from the first state, to the second state, and back to the first state and to change the signal provided by the RFID circuit from the first signal, to the second signal, and back to the first signal any number of times.

In some non-limiting embodiments, the RFID circuit may be configured to provide a first signal, such as during interrogation by an RFID reader device, when the electrical component is in a first state. In some non-limiting embodiments, a first signal provided by the RFID circuit may include data associated with an indication of a first state of container 650. In some non-limiting embodiments, data associated with a first state of container 650 may indicate a closed configuration. For example, data associated with a first state of container 650 may indicate a closed configuration for container 650. In such an example, data associated with a first state of container 650 may indicate that closure 660 is coupled to (e.g., attached to and/or the like) container body 652.

In some non-limiting embodiments, the first portion and the second portion of the electrical component may be decoupled when the electrical component is in a second state. For example, the first portion and the second portion of the electrical component may not be electrically coupled (e.g., not conductively coupled) when the electrical component is in a second state. In some non-limiting embodiments, first portion 612 of base component 610 may be decoupled from second portion 614 of base component 610 at attachment point 616, such that the first portion and the second portion of the electrical component are decoupled, when the electrical component is in a second state. For example, first portion 612 of base component 610 may be decoupled from second portion 614 of base component 610 at attachment point 616, such that there is gap between first portion 612 and second portion 614 of base component 610, where the gap is sufficiently wide, such that the first portion and the second portion of the electrical component are not electrically coupled. For example, closure 660 may be decoupled from (e.g., detached from and/or the like) container body 652, such that first portion 612 and second portion 614 of base component 610 may be decoupled, and the first portion and the second portion of the electrical component may be decoupled when the electrical component is in a second state. In some non-limiting embodiments, the RFID circuit may be configured to provide a second signal, such as during interrogation by an RFID reader device, when the electrical component is in a second state. In some non-limiting embodiments, the second signal provided by the RFID circuit may be different from the first signal provided by the RFID circuit.

In some non-limiting embodiments, a second signal provided by the RFID circuit may include data associated with an indication of a second state of container 650. In some non-limiting embodiments, data associated with a second state of container 650 may indicate an open configuration. For example, data associated with a second state of container 650 may indicate an open configuration for container 650. In such an example, data associated with a second state of container 650 may indicate that closure 660 has been decoupled from (detached from and/or the like) container body 652.

In some non-limiting embodiments, the first portion and the second portion of the electrical component may be recoupled after previously being decoupled when the electrical component is in the third state. For example, the first portion and the second portion of the electrical component may be electrically recoupled (e.g., conductively recoupled) after previously being electrically decoupled (e.g., conductively decoupled) when the electrical component is in a third state. In some non-limiting embodiments, first portion 612 and second portion 614 of base component 610 may be recoupled after previously being decoupled, such that the first portion and the second portion of the electrical component are recoupled after previously being decoupled, when the electrical component is in a third state. For example, closure 660 may be re coupled to (e.g., reattached to and/or the like) container body 652 after previously being detached from container body 652, such that first portion 612 and second portion 614 of base component 610 may be recoupled together, and the first portion and the second portion of the electrical component are recoupled together when the electrical component is in a third state. In some non-limiting embodiments, the first portion and the second portion of the electrical component may be decoupled and then recoupled to change the state of the electrical component from the first state, to the second state, to the third state and to change the signal provided by the RFID circuit from the first signal, to the second signal, to the third signal any number of times to provide any number of signals.

In some non-limiting embodiments, the RFID circuit may be configured to provide a third signal, such as during interrogation by an RFID reader device, when the electrical component is in a third state. In some non-limiting embodiments, the third signal provided by the RFID circuit may be different from both the first signal provided by the RFID circuit and the second signal provided by the RFID circuit. In some non-limiting embodiments, a third signal provided by the RFID circuit may include data associated with an indication of a third state of container 650. In some non-limiting embodiments, data associated with a third state of container 650 may indicate a reclosed configuration. For example, data associated with a third state of container 650 may indicate a reclosed configuration for container 650. In such an example, data associated with a third state of container 650 may indicate that closure 660 has been recoupled to (e.g., reattached to and/or the like) container body 652 after closure 660 had been previously decoupled from (e.g., detached from and/or the like) container body 652.

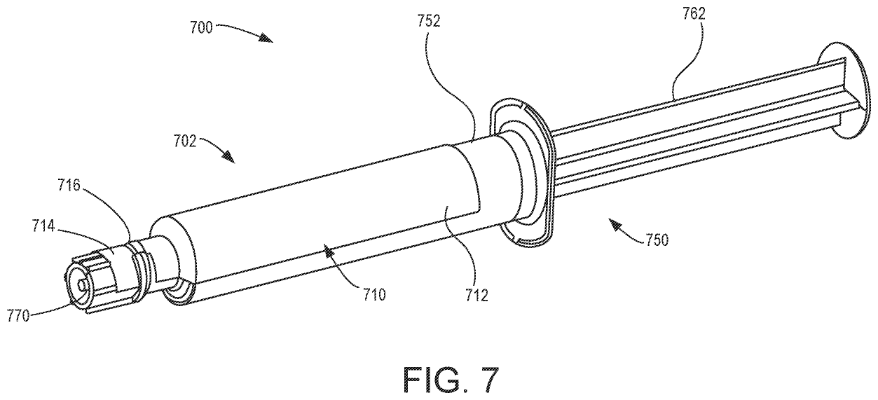

Referring to FIG. 7, FIG. 7 is a diagram of medical device assembly 700. As shown in FIG. 7, medical device assembly 700 includes RFID tag 702 positioned on syringe 750. In some non-limiting embodiments, RFID tag 702 may be attached to syringe 750 (e.g., via an adhesive and/or the like). In some non-limiting embodiments, RFID tag 702 may be the same as RFID tag 400 or RFID tag 500.

In some non-limiting embodiments, syringe 750 may have a size that is appropriate based on an application for which syringe 750 is designed. In some non-limiting embodiments, syringe 750 may have a shape that is appropriate based on an application which syringe 750 is designed. In some non-limiting embodiments, syringe 750 may include a material that is appropriate based on an application which syringe 750 is designed and that is appropriate based on the desired contents to be contained within syringe 750.

In some non-limiting embodiments, syringe 750 may include syringe body 752. In some non-limiting embodiments, syringe body 752 may have a size that is appropriate based on the contents to be contained within syringe body 752 and appropriate based on an application which syringe 750 is designed.

In some non-limiting embodiments, syringe 750 may include plunger 762. In some non-limiting embodiments, plunger 762 may have a size that is appropriate based on an application which syringe 750 is designed. For example, plunger 762 may have a size that corresponds to the size of the internal volume of syringe body 752. In some non-limiting embodiments, plunger 762 may have a shape that is appropriate based on an application which syringe 750 is designed. For example, plunger 762 may have a shape that corresponds to the shape of the internal volume of syringe body 752. In some non-limiting embodiments, plunger 762 may include a material that is appropriate based on an application that syringe 750 is designed. In some non-limiting embodiments, plunger 762 may be configured to be inserted through an opening and into the internal volume of syringe body 752.

In some non-limiting embodiments, syringe 750 may include closure 770. In some non-limiting embodiments, closure 770 may be a cap, a top, a seal, and/or the like. In some non-limiting embodiments, closure 770 may have a size that is appropriate based on an application which syringe 750 is designed. For example, closure 770 may have a size that is proportional to the size of syringe body 752. In some non-limiting embodiments, closure 770 may have a shape that is appropriate based on an application which syringe 750 is designed. For example, closure 770 may have a shape that corresponds to the shape of syringe body 752. In some non-limiting embodiments, closure 770 may be positioned on an end of syringe body 752. For example, closure 770 may be configured to be attached to syringe body 752 (e.g., via threading, luer attachment, and/or the like).

In some non-limiting embodiments, RFID tag 702 may be attached to syringe 750. In some non-limiting embodiments, RFID tag 702 may have a size that allows RFID tag 702 to be attached to syringe 750. In such an example, the size of RFID tag 702 may be proportional to the size of syringe 750. In some non-limiting embodiments, RFID tag 702 may have a shape that allows RFID tag 702 to be attached to syringe 750. In such an example, RFID tag 702 may have a shape that corresponds to syringe body 752 and/or to closure 770. As another example, RFID tag 702 may have a shape that is sufficient to include each of the components of RFID tag 702 that are to be positioned on RFID tag 702.

In some non-limiting embodiments, RFID tag 702 may include an RFID circuit, an electrical component, and base component 710. In some non-limiting embodiments, base component 710 may include attachment point 716 that is configured to allow for base component 710 to be separated into a first section and a second section.

In some non-limiting embodiments, RFID tag 702 may include the RFID circuit. In some non-limiting embodiments, the RFID circuit may be the same as RFID circuit 402. In some non-limiting embodiments, the RFID circuit may provide a signal based on a state of one or more components of RFID tag 702. For example, the RFID circuit may provide a first signal when one or more components of RFID tag 702 are in a first state, and provide a second signal when one or more components of RFID tag 702 are in a second state.

In some non-limiting embodiments, the electrical component may be the same as electrical component 404. In some non-limiting embodiments, the electrical component may include a first portion and a second portion. In some non-limiting embodiments, the first portion and the second portion of the electrical component may be the same as first portion 406 and second portion 408 of electrical component 404.

In some non-limiting embodiments, base component 710 may be the same as base component 410. In some non-limiting embodiments, base component 710 may be positioned on syringe 750. In some non-limiting embodiments, base component 710 may be attached to (e.g., via an adhesive and/or the like) syringe 750. For example, base component 710 may be attached to syringe body 752 and/or closure 770. As another example, base component 710 may be attached to syringe body 752 and/or plunger 762.

In some non-limiting embodiments, base component 710 may include first portion 712 and second portion 714. In some non-limiting embodiments, first portion 712 and second portion 714 of base component 710 may be the same as first portion 412 and second portion 414 of base component 410.

In some non-limiting embodiments, first portion 712 of base component 710 may be positioned on syringe body 752. For example, first portion 712 of base component 710 may be attached to (e.g., via an adhesive and/or the like) syringe body 752. In some non-limiting embodiments, first portion 712 of base component 710 may be positioned on closure 770. For example, first portion 712 of base component 710 may be attached to (e.g., via an adhesive and/or the like) closure 770. In some non-limiting embodiments, first portion 712 of base component 710 may be positioned on plunger 762. For example, first portion 712 of base component 710 may be attached to (e.g., via an adhesive and/or the like) plunger 762.

In some non-limiting embodiments, second portion 714 of base component 710 may be positioned on syringe body 752. For example, second portion 714 of base component 710 may be attached to (e.g., via an adhesive and/or the like) syringe body 752. In some non-limiting embodiments, second portion 714 of base component 710 may be positioned on closure 770. For example, second portion 714 of base component 710 may be attached to (e.g., via an adhesive and/or the like) closure 770. In some non-limiting embodiments, second portion 714 of base component 710 may be positioned on plunger 762. For example, second portion 714 of base component 710 may be attached to (e.g., via an adhesive and/or the like) plunger 762.

In some non-limiting embodiments, base component 710 may include attachment point 716 that separates first portion 712 of base component 710 from second portion 714 of base component 710. In some non-limiting embodiments, attachment point 716 may be the same as attachment point 416. In some non-limiting embodiments, attachment point 716 may be positioned at a gap between closure 770 and syringe body 752. For example, attachment point 716 may bridge the gap between closure 770 and syringe body 752. In some non-limiting embodiments, attachment point 716 may be positioned at a gap between plunger 762 and syringe body 752. For example attachment point 716 may bridge the gap between plunger 762 and syringe body 752.

In some non-limiting embodiments, the first portion and the second portion of the electrical component may be coupled together when the electrical component is in a first state. For example, the first portion and the second portion of the electrical component may be electrically coupled (e.g., conductively coupled) together when the electrical component is in a first state. In some non-limiting embodiments, first portion 712 and second portion 714 of base component 710 may be coupled together, such that the first portion and the second portion of the electrical component are coupled together, when the electrical component is in a first state. For example, closure 770 may be coupled to (e.g., attached to and/or the like) syringe body 752, such that first portion 712 and second portion 714 of base component 710 may be coupled together, and the first portion and the second portion of the electrical component are coupled together when the electrical component is in a first state. As another example, plunger 762 may be in an unused position relative to the syringe body 752, such that first portion 712 and second portion 714 of base component 710 may be coupled together, and the first portion and the second portion of the electrical component are coupled together when the electrical component is in a first state.

In some non-limiting embodiments, the first portion and the second portion of the electrical component may be recoupled after previously being decoupled when the electrical component is in the first state. For example, the first portion and the second portion of the electrical component may be electrically recoupled (e.g., conductively recoupled) after previously being electrically decoupled (e.g., conductively decoupled) when the electrical component is in a first state. In some non-limiting embodiments, first portion 712 and second portion 714 of base component 710 may be recoupled after previously being decoupled, such that the first portion and the second portion of the electrical component are recoupled after previously being decoupled, when the electrical component is in a first state. For example, closure 770 may be recoupled to (e.g., reattached to and/or the like) syringe body 752 after previously being removed from syringe body 752, such that first portion 712 and second portion 714 of base component 710 may be recoupled together, and the first portion and the second portion of the electrical component are recoupled together when the electrical component is in a first state. As another example, plunger 762 may be in a reuse position relative to syringe body 752 after previously being in a used position, such that first portion 712 and second portion 714 of base component 710 may be recoupled together, and the first portion and the second portion of the electrical component are recoupled together when the electrical component is in a first state. In some non-limiting embodiments, the first portion and the second portion of the electrical component may be decoupled and then recoupled to change the state of the electrical component from the first state, to the second state, and back to the first state and to change the signal provided by the RFID circuit from the first signal, to the second signal, and back to the first signal any number of times.

In some non-limiting embodiments, the RFID circuit may be configured to provide a first signal, such as during interrogation by an RFID reader device, when the electrical component is in a first state. In some non-limiting embodiments, a first signal provided by the RFID circuit may include data associated with an indication of a first state of syringe 750. In some non-limiting embodiments, data associated with a first state of syringe 750 may indicate a closed configuration. For example, data associated with a first state of syringe 750 may indicate a closed configuration for syringe 750. In such an example, data associated with a first state of syringe 750 may indicate that closure 770 is coupled to (e.g., attached to and/or the like) syringe body 752.

In some non-limiting embodiments, data associated with a first state of syringe 750 may indicate an unused configuration. For example, data associated with a first state of syringe 750 may indicate an unused configuration for syringe 750. In such an example, data associated with a first state of syringe 750 may indicate that plunger 762 is in an unused position relative to the syringe body 752 (e.g., plunger 762 has not been pushed into an internal volume of syringe body 752).

In some non-limiting embodiments, the first portion and the second portion of the electrical component may be decoupled when the electrical component is in a second state. For example, the first portion and the second portion of the electrical component may not be electrically coupled (e.g., not conductively coupled) when the electrical component is in a second state. In some non-limiting embodiments, first portion 712 of base component 710 may be decoupled from second portion 714 of base component 710 at attachment point 716, such that the first portion and the second portion of the electrical component are decoupled, when the electrical component is in a second state. For example, first portion 712 of base component 710 may be decoupled from second portion 714 of base component 710 at attachment point 716, such that there is a gap between first portion 712 and second portion 714 of base component 710, where the gap is sufficiently wide, such that the first portion and the second portion of the electrical component are not electrically coupled. For example, closure 770 may be decoupled from (e.g., detached from and/or the like) syringe body 752, such that first portion 712 and second portion 714 of base component 710 may be decoupled, and the first portion and the second portion of the electrical component may be decoupled when the electrical component is in a second state. As another example, plunger 762 may be in a used position relative to syringe body 752 such that first portion 712 and second portion 714 of base component 710 may be decoupled and the first portion and the second portion of the electrical component may be decoupled when the electrical component is in a second state. In some non-limiting embodiments, the RFID circuit may be configured to provide a second signal, such as during interrogation by an RFID reader device, when the electrical component is in a second state. In some non-limiting embodiments, the second signal provided by the RFID circuit may be different from the first signal provided by the RFID circuit.

In some non-limiting embodiments, a second signal provided by the RFID circuit may include data associated with an indication of a second state of syringe 750. In some non-limiting embodiments, data associated with a second state of syringe 750 may indicate an open configuration. For example, data associated with a second state of syringe 750 may indicate an open configuration for syringe 750. In such an example, data associated with a second state of syringe 750 may indicate that closure 770 has been decoupled from (e.g., detached from and/or the like) syringe body 752.

In some non-limiting embodiments, data associated with a second state of syringe 750 may indicate a used configuration. For examples, data associated with a second state of syringe 750 may indicate a used configuration for syringe 750. In such an example, data associated with a second state of syringe 750 may indicate that plunger 762 is in a used position relative to syringe body 752 (e.g., plunger 762 has been pushed into the internal volume of syringe body 752).

In some non-limiting embodiment, the first portion and the second portion of the electrical component may be recoupled after previously being decoupled when the electrical component is in the third state. For example, the first portion and the second portion of the electrical component may be electrically recoupled (e.g., conductively recoupled) after previously being electrically decoupled (e.g., conductively decoupled) when the electrical component is in a third state. In some non-limiting embodiments, first portion 712 and second portion 714 of base component 710 may be recoupled after previously being decoupled, such that the first portion and the second portion of the electrical component are recoupled after previously being decoupled, when the electrical component is in a third state. For example, closure 770 may be recoupled to (e.g., reattached to and/or the like) syringe body 752 after previously being removed from syringe body 752 such that first portion 712 and second portion 714 of base component 710 may be recoupled together and the first portion and the second portion of the electrical component are recoupled together when the electrical component is in a third state. In some non-limiting embodiments, the first portion and the second portion of the electrical component may be decoupled and then recoupled to change the state of the electrical component from the first state, to the second state, to the third state and to change the signal provided by the RFID circuit from the first signal, to the second signal, to the third signal any number of times to provide any number of signals.

In some non-limiting embodiments, the RFID circuit may be configured to provide a third signal, such as during interrogation by an RFID reader device, when the electrical component is in a third state. In some non-limiting embodiments, the third signal provided by the RFID circuit may be different from both the first signal provided by the RFID circuit and the second signal provided by The RFID circuit. In some non-limiting embodiments, a third signal provided by the RFID circuit may include data associated with an indication of a third state of syringe 750. In some non-limiting embodiments, data associated with a third state of syringe 750 may indicate a reclosed configuration. For example, data associated with a third state of syringe 750 may indicate a reclosed configuration for syringe 750. In such an example, data associated with a third state of syringe 750 may indicate that closure 770 has been recoupled to (e.g., reattached to and/or the like) syringe body 752 after closure 770 had been previously removed from syringe body 752.

In some non-limiting embodiments, data associated with a third state of syringe 750 may indicate a reused configuration. For example, data associated with a third state of syringe 750 may indicate a reused configuration for syringe 750. In such an example, data associated with a third state of syringe 750 may indicate that plunger 762 is in a reused position relative to syringe body 752 (e.g., plunger 762 has been pulled out of the internal volume of syringe body 752 after previously being pushed into the internal volume of syringe body 752).

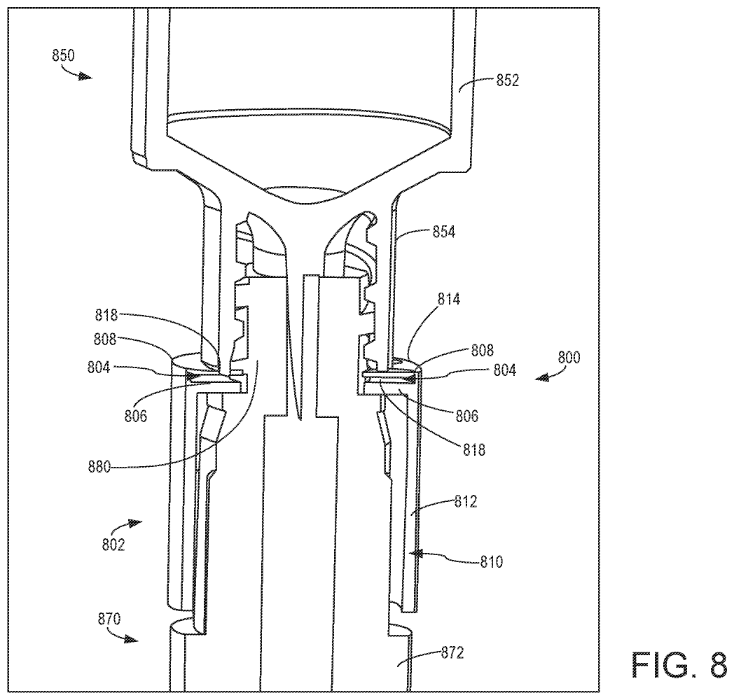

Referring to FIG. 8, FIG. 8 is a diagram of medical device assembly 800. As shown in FIG. 8, medical device assembly 800 includes RFID tag 802 positioned on and/or between syringe 850 and IV connector 870. For example, RFID tag 802 may be attached to syringe 850 and/or IV connector 870. In some non-limiting embodiments, RFID tag 802 may be the same as RFID tag 400 or RFID tag 500.

In some non-limiting embodiments, syringe 850 may have a size that is appropriate based on an application for which syringe 850 is designed. In some non-limiting embodiments, syringe 850 may have a shape that is appropriate based on an application which syringe 850 is designed. In some non-limiting embodiments, syringe 850 may include a material that is appropriate based on an application which syringe 850 is designed and that is appropriate based on the desired contents to be contained within syringe 850.

In some non-limiting embodiments, syringe 850 may include syringe body 852. In some non-limiting embodiments, syringe body 852 may have a size that is appropriate based on the contents to be contained within syringe body 852 and appropriate based on an application which syringe 850 is designed. In some non-limiting embodiments, syringe body 852 may have a shape that is appropriate based on the contents to be contained within syringe body 852 and appropriate based on an application which syringe 850 is designed. For example, the cross-section of syringe body 852 may be a circle, an oval, a rectangle, a square, and/or the like. In some non-limiting embodiments, syringe body 852 may include a material that is appropriate based on contents to be contained within syringe body 852.

In some non-limiting embodiments, syringe body 852 may include connection tip 854 at an end. In some non-limiting embodiments, connection tip 854 of syringe body 852 may be threaded. In some non-limiting embodiments, connection tip 854 of syringe body 852 may include a luer connector. In some non-limiting embodiments, connection tip 854 of syringe body 852 may be configured to attach to a connector (e.g., IV connector 870).

In some non-limiting embodiments, the IV assembly may include IV connector 870. In some non-limiting embodiments, IV connector 870 may have a size that is appropriate based on an application for which IV connector 870 is designed. In some non-limiting embodiments, IV connector 870 may have a shape that is appropriate based on an application which IV connector 870 is designed. In some non-limiting embodiments, IV connector 870 may include a material that is appropriate based on an application which IV connector 870 is designed.

In some non-limiting embodiments, IV connector 870 may include connector body 872. In some non-limiting embodiments, connector body 872 may have a size that is appropriate based on the contents to be contained within connector body 872 and appropriate based on an application which IV connector 870 is designed. In some non-limiting embodiments, connector body 872 may have a shape that is appropriate based on an application which IV connector 870 is designed. For example, the cross-section of connector body 872 may be a circle, an oval, a rectangle, a square, and/or the like. In some non-limiting embodiments, connector body 872 may include a material that is appropriate based on contents to be contained within connector body 872.

In some non-limiting embodiments, connector body 872 may include connection tip 880 at an end of connector body 872. In some non-limiting embodiments, connection tip 880 of connector body 872 may be threaded. In some non-limiting embodiments, connection tip 880 of connector body 872 may include a luer connector. In some non-limiting embodiments, connection tip 880 of connector body 872 may be configured to couple to connection tip 854 of syringe body 852. For example, connection tip 880 of connector body 872 may be configured to connect to connection tip 854 of syringe body 852 (e.g., via threaded attachment, luer attachment, and/or the like).

In some non-limiting embodiments, RFID tag 802 may be attached to syringe 850 and/or IV connector 870. In some non-limiting embodiments, RFID tag 802 may have a size that allows RFID tag 802 to be attached to syringe 850 and/or IV connector 870. In such an example, the size of RFID tag 802 may be proportional to the size of syringe 850 and/or IV connector 870.

In some non-limiting embodiments, RFID tag 802 may have a shape that allows RFID tag 802 to be positioned between (e.g., attached to and/or the like) syringe 850 and IV connector 870. In such an example, RFID tag 802 may have a shape that corresponds to IV connector 870 and/or syringe 850.

In some non-limiting embodiments, RFID tag 802 may include an RFID circuit, electrical component 804, and base component 810.

In some non-limiting embodiments, RFID tag 802 may include the RFID circuit. In some non-limiting embodiments, the RFID circuit may be the same as RFID circuit 402. In some non-limiting embodiments, the RFID circuit may provide a signal based on a state of one or more components of RFID tag 802. For example, the RFID circuit may provide a first signal when one or more components of RFID tag 802 are in a first state, and provide a second signal when one or more components of RFID tag 802 are in a second state.

In some non-limiting embodiments, electrical component 804 may be the same as electrical component 404. In some non-limiting embodiments, electrical component 804 may include a first portion 806 and a second portion 808. In some non-limiting embodiments, first portion 806 of electrical component 804 may be first conductive tab 806 and second portion 808 of electrical component 804 may be second conductive tab 808. In some non-limiting embodiments, first conductive tab 806 and second conductive tab 808 of electrical component 804 may be separated by gap 818. In some non-limiting embodiments, first conductive tab 806 and second conductive tab 808 of electrical component 804 may be separated by gap 818 that is of a sufficient width, such that first conductive tab 806 and second conductive tab 808 of electrical component 804 are decoupled. For example, first conductive tab 806 and second conductive tab 808 of electrical component 804 may be separated by gap 818 that is of a sufficient width, such that first conductive tab 806 and second conductive tab 808 of electrical component 804 are not electrically connected. In some non-limiting embodiments, first portion 806 and second portion 808 of electrical component 804 (e.g., first conductive tab 806 and second conductive tab 808) may be the same as first portion 406 and second portion 408 of electrical component 404. In some non-limiting embodiments, first portion 806 and second portion 808 of electrical component 804 may be separable based on one or more mechanical components. For example, first portion 806 and second portion 808 of electrical component 804 may be separable based on the disconnection of syringe 850 and IV connector 870. In some non-limiting embodiments, first portion 806 of electrical component 804 may be connected (e.g., electrically connected) to the RFID circuit.

In some non-limiting embodiments, base component 810 may be the same as base component 410. In some non-limiting embodiments, base component 810 may be positioned on and/or between syringe 850 and IV connector 870. In some non-limiting embodiments, base component 810 may be attached to (e.g., via an adhesive and/or the like) syringe 850 and/or IV connector 870.

In some non-limiting embodiments, base component 810 may include first portion 812 and second portion 814. In some non-limiting embodiments, first portion 812 and second portion 814 of base component 810 may be the same as first portion 412 and second portion 414 of base component 410.

In some non-limiting embodiments, first portion 812 of base component 810 may be positioned on IV connector 870. For example, first portion 812 of base component 810 may be attached to (e.g., via an adhesive and/or the like) IV connector 870. In some non-limiting embodiments, first portion 812 of base component 810 may be positioned on syringe 850. For example, first portion 812 of base component 810 may be attached to (e.g., via an adhesive and/or the like) syringe 850.

In some non-limiting embodiments, second portion 814 of base component 810 may be positioned on IV connector 870. For example, second portion 814 of base component 810 may be attached to (e.g., via an adhesive and/or the like) IV connector 870. In some non-limiting embodiments, second portion 814 of base component 810 may be positioned on syringe 850. For example, second portion 814 of base component 810 may be attached to (e.g., via an adhesive and/or the like) syringe 850.

In some non-limiting embodiments, a portion of first portion 812 and second portion 814 of base component 810 may be materially connected, and another portion of first portion 812 and second portion 814 of base component 810 may be separated by gap 818.

In some non-limiting embodiments, first portion 806 and second portion 808 of electrical component 804 may be coupled together when electrical component 804 is in a first state. For example, first portion 806 and second portion 808 of electrical component 804 may be electrically coupled (e.g., conductively coupled) together when electrical component 804 is in a first state. In some non-limiting embodiments, first portion 812 and second portion 814 of base component 810 may be coupled together, such that first portion 806 and second portion 808 of electrical component 804 are coupled together, when electrical component 804 is in a first state. For example, syringe 850 may be coupled to (e.g., attached to and/or the like) IV connector 870, such that first portion 812 and second portion 814 of base component 810 may be coupled together, and first portion 806 and second portion 808 of electrical component 804 are coupled together when electrical component 804 is in a first state.

In some non-limiting embodiments, first portion 806 and second portion 808 of electrical component 804 may be recoupled after previously being decoupled when electrical component 804 is in the first state. For example, first portion 806 and second portion 808 of electrical component 804 may be electrically recoupled (e.g., conductively recoupled) after previously being electrically decoupled (e.g., conductively decoupled) when electrical component 804 is in a first state. In some non-limiting embodiments, first portion 812 and second portion 814 of base component 810 may be recoupled after previously being decoupled, such that first portion 806 and second portion 808 of electrical component 804 are recoupled after previously being decoupled, when electrical component 804 is in a first state. For example, syringe 850 may be re coupled to (e.g., reattached to and/or the like) IV connector 870, after previously being decoupled from (e.g., detached from and/or the like) IV connector 870, such that first portion 812 and second portion 814 of base component 810 may be recoupled together, and first portion 806 and second portion 808 of electrical component 804 are recoupled together when electrical component 804 is in a first state. In some non-limiting embodiments, first portion 806 and second portion 808 of electrical component 804 may be decoupled and then recoupled to change the state of electrical component 804 from the first state, to the second state, and back to the first state and to change the signal provided by the RFID circuit from the first signal, to the second signal, and back to the first signal any number of times.

In some non-limiting embodiments, the RFID circuit may be configured to provide a first signal, such as during interrogation by an RFID reader device, when electrical component 804 is in a first state. In some non-limiting embodiments, a first signal provided by the RFID circuit may include data associated with an indication of a first state of syringe 850 and/or IV connector 870. For example, data associated with a first state of syringe 850 and/or IV connector 870 may indicate a coupled configuration. For example, data associated with a first state of syringe 850 and/or IV connector 870 may indicate a coupled configuration for syringe 850 and/or IV connector 870. For example, data associated with a first state of syringe 850 and/or IV connector 870 may indicate that syringe 850 is coupled to IV connector 870. In such an example, data associated with a first state of syringe 850 and/or IV connector 870 may indicate that a connection tip 854 of syringe body 852 (e.g., via threading, a luer connector, and/or the like) is connected to connection tip 880 of connector body 872 (e.g., via threading, a luer connector, and/or the like).

In some non-limiting embodiments, first portion 806 and second portion 808 of electrical component 804 may be decoupled when electrical component 804 is in a second state. For example, first portion 806 and second portion 808 of electrical component 804 may not be electrically coupled (e.g., not conductively coupled) when electrical component 804 is in a second state. In some non-limiting embodiments, first portion 812 of base component 810 may be decoupled from second portion 814 of base component 810, such that first portion 806 and second portion 808 of electrical component 804 are decoupled, when electrical component 804 is in a second state. For example, first portion 812 of base component 810 may be decoupled from second portion 814 of base component 810, such that there is gap 818 between first portion 812 and second portion 814 of base component 810, where the gap 818 is sufficiently wide, such that first portion 806 and second portion 808 of electrical component 804 are not electrically coupled. For example, syringe 850 may be de coupled from (e.g., detached from and/or the like) IV connector 870, such that first portion 812 and second portion 814 of base component 810 may be decoupled, and first portion 806 and second portion 808 of electrical component 804 may be decoupled when electrical component 804 is in a second state. In some non-limiting embodiments, the RFID circuit may be configured to provide a second signal, such as during interrogation by an RFID reader device, when electrical component 804 is in a second state. In some non-limiting embodiments, the second signal provided by the RFID circuit may be different from the first signal provided by the RFID circuit.

In some non-limiting embodiments, a second signal provided by the RFID circuit may include data associated with an indication of a second state of syringe 850 and/or IV connector 870. In some non-limiting embodiments, data associated with a second state of syringe 850 and/or IV connector 870 may indicate a decoupled configuration. For example, data associated with a second state of syringe 850 and/or IV connector 870 may indicate a decoupled configuration for syringe 850 and IV connector 870. For example, data associated with a second state of syringe 850 and/or IV connector 870 may indicate that syringe 850 is decoupled from (e.g., detached from and/or the like) IV connector 870. In such an example, data associated with a second state of syringe 850 and/or IV connector 870 may indicate that connection tip 854 of syringe body 852 (e.g., via disengagement of threading, a luer connector and/or the like) is not connected to connection tip 880 of connector body 872 (e.g., via disengagement of threading, a luer connector and/or the like).

In some non-limiting embodiments, first portion 806 and second portion 808 of electrical component 804 may be recoupled after previously being decoupled when electrical component 804 is in the third state. For example, first portion 806 and second portion 808 of electrical component 804 may be electrically recoupled (e.g., conductively recoupled) after previously being electrically decoupled (e.g., conductively decoupled) when electrical component 804 is in a third state. In some non-limiting embodiments, first portion 812 and second portion 814 of base component 810 may be recoupled after previously being decoupled, such that first portion 806 and second portion 808 of electrical component 804 are recoupled after previously being decoupled, when electrical component 804 is in a third state. For example, syringe 850 may be recoupled to (e.g., reattached to and/or the like) IV connector 870, after previously being decoupled from (e.g., detached from) IV connector 870, such that first portion 812 and second portion 814 of base component 810 may be recoupled together, and first portion 806 and second portion 808 of electrical component 804 are recoupled together when electrical component 804 is in a third state. In some non-limiting embodiments, first portion 806 and second portion 808 of electrical component 804 may be decoupled and then recoupled to change the state of electrical component 804 from the first state, to the second state, to the third state and to change the signal provided by the RFID circuit from the first signal, to the second signal, to the third signal any number of times to provide any number of signals.

In some non-limiting embodiments, the RFID circuit may be configured to provide a third signal, such as during interrogation by an RFID reader device, when electrical component 804 is in a third state. In some non-limiting embodiments, the third signal provided by the RFID circuit may be different from both the first signal provided by the RFID circuit and the second signal provided by the RFID circuit. In some non-limiting embodiments, a third signal provided by the RFID circuit may include data associated with an indication of a third state of syringe 850 and/or IV connector 870. In some non-limiting embodiments, data associated with a third state of syringe 850 and/or IV connector 870 may indicate a recoupled configuration. For example, data associated with a third state of syringe 850 and/or IV connector 870 may indicate a recoupled configuration for syringe 850 and IV connector 870. In such an example, data associated with a third state of syringe 850 and/or IV connector 870 may indicate that connection tip 854 of syringe body 852 (e.g., via threading, a luer connector, and/or the like) has been recoupled to (e.g., reattached to and/or the like) connection tip 880 of connector body 872 (e.g., via threading, a luer connector, and/or the like) after previously being decoupled (e.g., detached and/or the like).

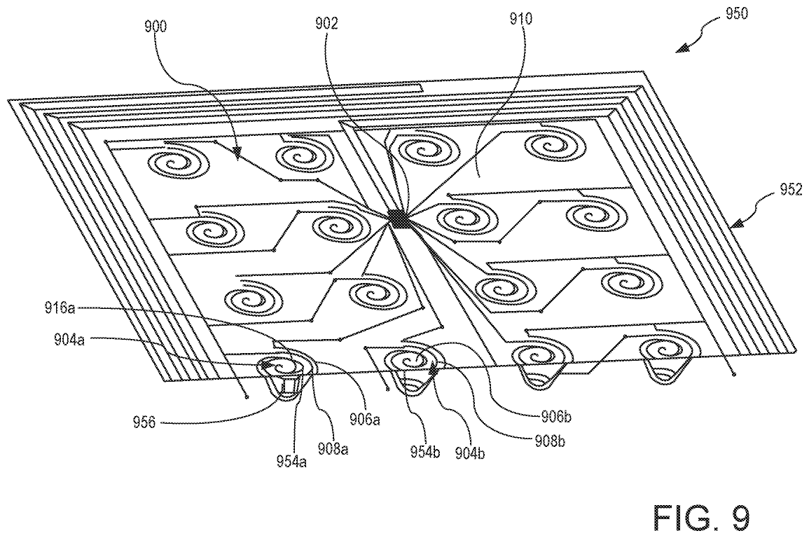

Referring to FIG. 9, FIG. 9 is a diagram of medical device assembly 950. As shown in FIG. 9, medical device assembly 950 includes RFID tag 900 positioned on medication packaging 952. In some non-limiting embodiments, RFID tag 900 may be attached to medication packaging 952 (e.g., via an adhesive and/or the like). In some non-limiting embodiments, RFID tag 900 may be the same as RFID tag 400 or RFID tag 500.

In some non-limiting embodiments, medication packaging 952 may have a size that is appropriate based on an application for which medication packaging 952 is designed. For example, medication packaging 952 may have a size that is sufficient to contain medication 956 within. In some non-limiting embodiments, medication packaging 952 may have a shape that is appropriate based on an application which medication packaging 952 is designed. In some non-limiting embodiments, medication packaging 952 may include a material that is appropriate based on an application which medication packaging 952 is designed. For example, the material of medication packaging 952 may include plastic, metal, and/or the like. In some non-limiting embodiments, medication packaging 952 may be medication blister pack 952.

In some non-limiting embodiments, medication packaging 952 may include medication web 954a. In some non-limiting embodiments, medication web 954a may be positioned on medication packaging 952. For example, medication web 954a may be materially attached to medication packaging 952. In some non-limiting embodiments, medication web 954a may be sealed by medication packaging 952. In some non-limiting embodiments, medication web 954a may have a size that is appropriate based on an application which medication packaging 952 is designed. For example, medication web 954a may have a size, such that medication 956 may be positioned within medication web 954a. In some non-limiting embodiments, medication web 954a may have a shape that is appropriate based on an application which medication packaging 952 is designed. For example, medication web 954a may have a shape, such that medication 956 may be positioned within medication web 956. In some non-limiting embodiments, medication web 954a may include a material that is appropriate based on an application which medication packaging 952 is designed. For example, medication web 954a may include a material based on medication 956 that is contained within medication web 954a. For example, medication web 954a may include plastic.

In some non-limiting embodiments, medication packaging 952 may include medication 956. For example, medication 956 may be contained within medication web 954a (e.g., sealed within medication web 954a by medication packaging 952). In some non-limiting embodiments, medication packaging 952 may include a plurality of medication webs 954a, 954b, where medication 956 is positioned within each of the medication webs of a plurality of medication webs 954a, 954b.

In some non-limiting embodiments, RFID tag 900 may be attached to medication packaging 952 (e.g., via an adhesive and/or the like). In some non-limiting embodiments, RFID tag 900 may have a size that allows RFID tag 900 to be attached to medication packaging 952. In such an example, RFID tag 900 may have a size that is proportional to a size of medication packaging 952. In some non-limiting embodiments, RFID tag 900 may have a shape that allows RFID tag 900 to be attached to medication packaging 952.

In some non-limiting embodiments, RFID tag 900 may include RFID circuit 902, a plurality of electrical components 904a, 904b, and base component 910. In some non-limiting embodiments, base component 910 may include a plurality of attachment points 916a, 916b, each of which is positioned over medication web 954a, 954b, and each attachment point 916a, 916b being configured to allow for base component 910 to be punctured, such as to unseal medication webs 954a, 954b to allow medication 956 to be removed from medication webs 954a, 954b.

In some non-limiting embodiments, RFID tag 900 may include RFID circuit 902. In some non-limiting embodiments, RFID circuit 902 may be the same as RFID circuit 402. In some non-limiting embodiments, RFID circuit 902 may provide a signal based on a state of one or more components of RFID tag 900. For example, RFID circuit 902 may provide a first signal when one or more components of RFID tag 900 are in a first state, and provide a second signal when one or more components of RFID tag 900 are in a second state.

In some non-limiting embodiments, a plurality of electrical components 904a, 904b may include first electrical component 904a. In some non-limiting embodiments, first electrical component 904a may be the same as electrical component 404. In some non-limiting embodiments, first electrical component 904a may have a size that corresponds to a size of medication web 954a. In some non-limiting embodiments, first electrical component 904a may be positioned over medication web 954a. For example, first electrical component 904a may be positioned on a portion of base component 910 that seals medication web 954a.

In some non-limiting embodiments, first electrical component 904a may include a first portion 906a and a second portion 908a. In some non-limiting embodiments, first portion 906a and second portion 908a of first electrical component 904a may be the same as first portion 406 and second portion 408 of electrical component 404. In some non-limiting embodiments, first portion 906a and second portion 908a of first electrical component 904a may be positioned over medication web 954a. For example, first portion 906a and second portion 908a of first electrical component 904a may be positioned on a portion of base component 910 that seals medication web 954a. In some non-limiting embodiments, a plurality of electrical components 904a, 904b may include second electrical component 904b. In some non-limiting embodiments, second electrical component 904b may be the same as electrical component 404. In some non-limiting embodiments, second electrical component 904b may have a size that is appropriate based on an application for which RFID tag 900 is designed. For example, second electrical component 904b may have a size, such that second electrical component 904b may be positioned on base component 910. In such an example, second electrical component 904b may have a size that corresponds to a size of medication web 954b.

In some non-limiting embodiments, second electrical component 904b may be positioned over medication web 954b. For example, second electrical component 904b may be positioned on a portion of base component 910 that seals medication web 954b.

In some non-limiting embodiments, second electrical component 904b may include first portion 906b and second portion 908b. In some non-limiting embodiments, first portion 906b and second portion 908b of second electrical component 904b may be the same as first portion 406 and second portion 408 of electrical component 404. In some non-limiting embodiments, first portion 906b and second portion 908b of second electrical component 904b may be positioned over medication web 954b. For example first portion 906b and second portion 908b of second electrical component 904b may be positioned on a portion of base component 910 that seals medication web 954b.

It can be readily envisioned that additional electrical components, such as a third electrical component, fourth electrical component, fifth electrical component, and/or the like, with the same characteristics as a plurality of electrical components 904a, 904b, may be included in RFID tag 900. For example, RFID tag 900 may include at least two, or at least three, or at least four, or at least five electrical components 904, where each electrical component 904 is positioned over a medication web of a plurality of medication webs 954a, 954b, such as on a portion of base component 910 that seals a medication web of plurality of medication webs 954a, 954b.

In some non-limiting embodiments, base component 910 may be the same as base component 410. In some non-limiting embodiments, a plurality of attachment points 916a, 916b may include first attachment point 916a. In some non-limiting embodiments, first attachment point 916a may be the same as attachment point 416. In some non-limiting embodiments, first attachment point 916a may be positioned over medication web 954a. For example, first attachment point 916a may be positioned on a portion of base component 910 that seals medication web 954a. In some non-limiting embodiments, first attachment point 916a may be configured to allow base component 910 to be punctured at the portion that seals medication web 954a.

In some non-limiting embodiments, a plurality of attachment points 916a, 916b may include second attachment point 916b. In some non-limiting embodiments, second attachment point 916b may be the same as attachment point 416. In some non-limiting embodiments, second attachment point 916b may be positioned over medication web 954b. For example, second attachment point 916b may be positioned on a portion of base component 910 that seals medication web 954b. In some non-limiting embodiments, second attachment point 916b may be configured to allow base component 910 to be punctured at the portion that seals medication web 954b.

In some non-limiting embodiments, first portion 906a and second portion 908a of first electrical component 904a may be coupled together when first electrical component 904a is in a first state. For example, first portion 906a and second portion 908a of first electrical component 904a may be electrically coupled (e.g., conductively coupled) together when first electrical component 904a is in a first state. In some non-limiting embodiments, base component 910 has not been punctured, such that medication web 954a is sealed, such that first portion 906a and second portion 908a of first electrical component 904a are coupled together, when first electrical component 904a is in a first state.

In some non-limiting embodiments, first portion 906b and second portion 908b of second electrical component 904b may be coupled together when second electrical component 904b is in a first state. For example, first portion 906b and second portion 908b of second electrical component 904b may be electrically coupled (e.g., conductively coupled) together when second electrical component 904b is in a first state. In some non-limiting embodiments, base component 910 has not been punctured such that medication web 954b is sealed, such that first portion 906b and second portion 908b of second electrical component 904b are coupled together, when second electrical component 904b is in a first state.

In some non-limiting embodiments, RFID circuit 902 may be configured to provide a first signal, such as during interrogation by an RFID reader device, when first electrical component 904a and/or second electrical component 904b is in a first state. In some non-limiting embodiments, a first signal provided by RFID circuit 902 may include data associated with an indication of a first state of medication packaging 952. In some non-limiting embodiments, data associated with a first state of medication packaging 952 may indicate a sealed configuration. For example, data associated with a first state of medication packaging 952 may indicate a sealed configuration of medication packaging 952. In such an example, data associated with a first state of medication packaging 952 may indicate that base component 910 has not been punctured at any of a plurality of attachment points 916a, 916b to unseal any of a plurality of medication webs 954a, 954b. In another example, data associated with a first state of medication packaging 952 may indicate base component 910 has not been punctured at first attachment point 916a to unseal medication web 954a.

In some non-limiting embodiments, first portion 906a and second portion 908a of first electrical component 904a may be decoupled when first electrical component 904a is in a second state. For example, first portion 906a and second portion 908a of first electrical component 904a may not be electrically coupled (e.g., not conductively coupled) when first electrical component 904a is in a second state. In some non-limiting embodiments, base component 910 may be punctured at first attachment point 916a, such that medication web 954a is unsealed, such that first portion 906a and second portion 908a of first electrical component 904a are decoupled, when first electrical component 904a is in a second state.

In some non-limiting embodiments, first portion 906b and second portion 908b of second electrical component 904b may be decoupled when second electrical component 904b is in a second state. For example, first portion 906b and second portion 908b of second electrical component 904b may not be electrically coupled (e.g., not conductively coupled) when second electrical component 904b is in a second state. In some non-limiting embodiments, base component 910 may be punctured at second attachment point 916b, such that medication web 954b is unsealed, such that first portion 906b and second portion 908b of second electrical component 904b are decoupled, when second electrical component 904b is in a second state.

In some non-limiting embodiments, RFID circuit 902 may be configured to provide a second signal, such as during interrogation by an RFID reader device, when first electrical component 904a and/or second electrical component 904b is in a second state. In some non-limiting embodiments, the second signal provided by RFID circuit 902 may be different from the first signal provided by RFID circuit 902.

In some non-limiting embodiments, a second signal provided by RFID circuit 902 may include data associated with an indication of a second state of medication packaging 952. In some non-limiting embodiments, data associated with a second state of medication packaging 952 may indicate an unsealed configuration. For example, data associated with a second state of medication packaging 952 may indicate an unsealed configuration of medication packaging 952. In such an example, data associated with a second state of medication packaging 952 may indicate that base component 910 has been punctured at any of a plurality of attachment points 916a, 916b to unseal any of a plurality of medication webs 954a, 954b. In another example, data associated with a second state of medication packaging 952 may indicate base component 910 has been punctured at first attachment point 916a to unseal medication web 954a.

In some non-limiting embodiments, first portion 906b and second portion 908b of second electrical component 904b may be coupled together when second electrical component 904b is in a third state. For example, first portion 906b and second portion 908b of second electrical component 904b may be electrically coupled (e.g., conductively coupled) together when second electrical component 904b is in a third state. In some non-limiting embodiments, base component 910 has not been punctured such that medication web 954b is sealed, such that first portion 906b and second portion 908b of second electrical component 904b are coupled together, when second electrical component 904b is in a third state.

In some non-limiting embodiments, RFID circuit 902 may be configured to provide a third signal, such as during interrogation by an RFID reader device, when second electrical component 904b is in a third state. In some non-limiting embodiments, the third signal provided by RFID circuit 902 may be different from both the first signal and the second signal provided by RFID circuit 902.

In some non-limiting embodiments, a third signal provided by RFID circuit 902 may include data associated with an indication of a third state of medication packaging 952. In some non-limiting embodiments, data associated with a third state of medication packaging 952 may indicate a sealed configuration. For example, data associated with a third state of medication packaging 952 may indicate a sealed configuration of medication packaging 952. For example, data associated with a third state of medication packaging 952 may indicate base component 910 has not been punctured at second attachment point 916b to unseal medication web 954b.

In some non-limiting embodiments, first portion 906b and second portion 908b of second electrical component 904b may be decoupled when second electrical component 904b is in a fourth state. For example, first portion 906b and second portion 908b of second electrical component 904b may not be electrically coupled (e.g., not conductively coupled) when second electrical component 904b is in a fourth state. In some non-limiting embodiments, base component 910 may be punctured at second attachment point 916b, such that medication web 954b is unsealed, such that first portion 906b and second portion 908b of second electrical component 904b are decoupled, when second electrical component 904b is in a fourth state.

In some non-limiting embodiments, RFID circuit 902 may be configured to provide a fourth signal, such as during interrogation by an RFID reader device, when second electrical component 904b is in a fourth state. In some non-limiting embodiments, the fourth signal provided by RFID circuit 902 may be different from the first signal, the second signal, and the third signal provided by RFID circuit 902.

In some non-limiting embodiments, a fourth signal provided by RFID circuit 902 may include data associated with an indication of a fourth state of medication packaging 952. In some non-limiting embodiments, data associated with a fourth state of medication packaging 952 may indicate an unsealed configuration. For example, data associated with a fourth state of medication packaging 952 may indicate an unsealed configuration of medication packaging 952. For example, data associated with a fourth state of medication packaging 952 may indicate base component 910 has been punctured at second attachment point 916b to unseal medication web 954b.

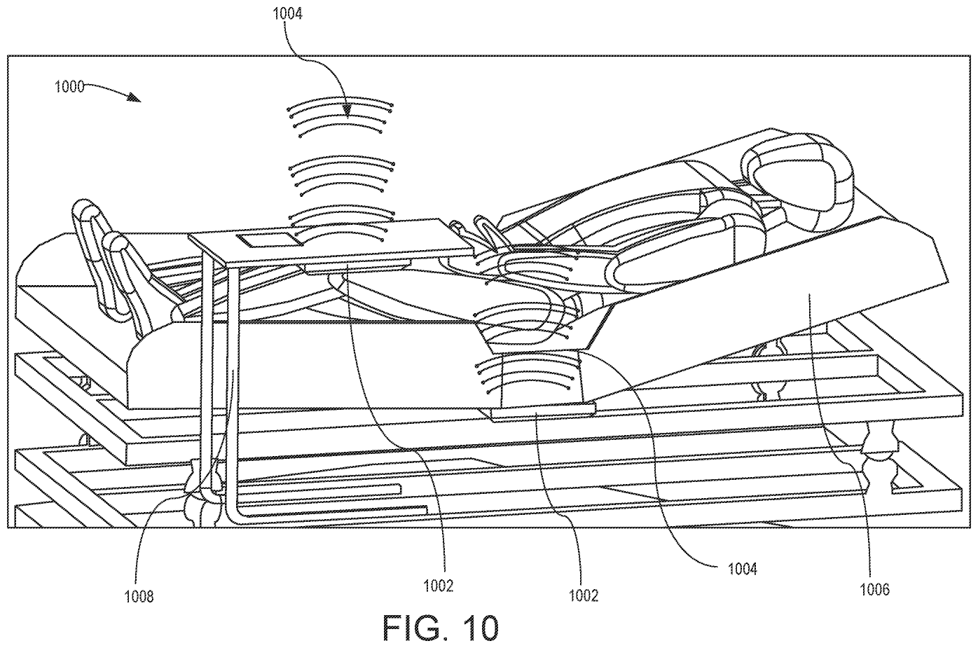

Referring to FIG. 10, FIG. 10 is a diagram of non-limiting positions for RFID reader device 1002. In some non-limiting embodiments, area 1000 may include RFID reader device 1002. In some non-limiting embodiments, RFID reader device 1002 may be the same as or similar to RFID reader device 104. In some non-limiting embodiments, RFID reader device 1002 may be positioned on bed 1006. For example, RFID reader device 1002 may be attached to bed 1006 (e.g., via an adhesive, fastener, and/or the like). As another example, RFID reader device 1002 may form part of bed 1006, such that RFID reader device 1002 and bed 1006 are materially connected.

In some non-limiting embodiments, RFID reader device 1002 may be positioned on side table 1008. For example, RFID reader device 1002 may be attached to side table 1008 (e.g., via an adhesive, fastener, and/or the like). In another example, RFID reader device 1002 may form part of side table 1008, such that RFID reader device 1002 and side table 1008 are materially connected.

It can be readily envisioned that RFID reader device 1002 may be positioned elsewhere in area 1000. For example, RFID reader device 1002 may be positioned on a patient, such as a wristlet, attached to a gown, and/or the like. In another example, RFID reader device 1002 may be positioned on a wall, a ceiling, and/or a floor of area 1000 and/or on medical equipment present in area 1000.

In some non-limiting embodiments, RFID reader device 1002 may receive data from an RFID tag (e.g., RFID tag 400 and/or RFID tag 500) and transmit notification 1004, based on the data received. For example, RFID reader device 1002 may be cloud connected. In some non-limiting embodiments, RFID reader device 1002 may receive data from an RFID tag (e.g., RFID tag 400 and/or RFID tag 500) and transmit notification 1004, based on the data received, via an email, an instant message, and/or the like. For example, RFID reader device may transmit notification 1004, based on the data received, to a clinician, an outpatient office, a patient, a spouse, an insurance provider, and/or the like.

Although non-limiting embodiments have been described in detail for the purpose of illustration and description, it is to be understood that such detail is solely for that purpose and that embodiments are not limited to the disclosed embodiments, but, on the contrary, are intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect. In fact, many of these features can be combined in ways not specifically recited in the claims and/or disclosed in the specification. Although each dependent claim listed below may directly depend on only one claim, the disclosure of possible implementations includes each dependent claim in combination with every other claim in the claim set.

1-22. (canceled)

23. A system comprising:
a medical device; and
a radio frequency identification (RFID) tear tag attached to the medical device, wherein the RFID tear tag comprises:
an RFID circuit; and
an electrical component electrically connected to the RFID circuit, wherein the electrical component comprises a first portion and a second portion, and wherein the first portion and the second portion are configured to be separable,
wherein, when the electrical component is in a first state, the first portion and the second portion of the electrical component are electrically coupled and the RFID circuit is configured to provide a first signal, and
wherein, when the electrical component is in a second state, the first portion and the second portion of the electrical component are not electrically coupled and the RFID circuit is configured to provide a second signal.

24. The system of claim 23, wherein, when the first portion and the second portion of the electrical component are electrically coupled, the first portion and the second portion of the electrical component are conductively coupled.

25. The system of claim 23, wherein the electrical component comprises an electrical conductor.

26. The system of claim 25, wherein the electrical conductor comprises an electrically conductive trace.

27. The system of claim 23, further comprising:
a base component,
wherein the RFID circuit and the electrical component are positioned on the base component.

28. The system of claim 27, wherein the base component comprises a perforation, and wherein the first portion and the second portion are configured to be separable based on the perforation of the base component.

29. The system of claim 23, wherein, when the electrical component is in the first state, the first portion and the second portion are coupled together, and wherein when the electrical component is in the second state the first portion and the second portion cannot be recoupled.

30. The system of claim 23, wherein the first portion and the second portion are configured to be separable independent of an additional mechanical component.

31. The system of claim 23, wherein the electrical component is a first electrical component, and further comprises:
a second electrical component electrically connected to the RFID circuit, wherein the second electrical component comprises an additional first portion and an additional second portion, and wherein the additional first portion of the second electrical component and the additional second portion of the second electrical component are configured to be separable; and
wherein the additional first portion is separated from the additional second portion, the RFID circuit is configured to provide a third signal, wherein the third signal is different from the first signal and the second signal.

32. The system of claim 23, wherein, when the electrical component is in the first state, the RFID circuit is configured to provide the first signal during interrogation by an RFID reader device.

33. The system of claim 23, wherein, when the electrical component is in the second state, the RFID circuit is configured to provide the second signal during interrogation by an RFID reader device, wherein the second signal is different from the first signal.

34. The system of claim 23, wherein the medical device comprises a single-use medical device.

35. A medical device assembly comprising:
a container having a closure;
a radio frequency identification (RFID) circuit; and
an electrical component electrically connected to the RFID circuit, wherein the electrical component comprises a first portion and a second portion, and wherein the first portion and the second portion are configured to be separable, and
wherein the RFID circuit and the electrical component are positioned on the container,
wherein, when the electrical component is in a first state, the first portion and the second portion of the electrical component are electrically coupled and the RFID circuit is configured to provide a first signal during interrogation by an RFID reader device, and
wherein, when the electrical component is in a second state, the first portion and the second portion of the electrical component are not electrically coupled and the RFID circuit is configured to provide a second signal during interrogation by an RFID reader device, wherein the second signal is different from the first signal.

36. The medical device assembly of claim 35, wherein, when the first portion and the second portion of the electrical component are electrically coupled, the first portion and the second portion of the electrical component are conductively coupled.

37. The medical device assembly of claim 35, wherein the electrical component comprises an electrical conductor.

38. The medical device assembly of claim 37, wherein the electrical conductor comprises an electrically conductive trace.

39. The medical device assembly of claim 35, further comprising:
a base component,
wherein the RFID circuit and the electrical component are positioned on the base component, and
wherein the base component is positioned on the closure of the container.

40. The medical device assembly of claim 39, wherein the base component comprises a perforation, and wherein the first portion and the second portion are configured to be separable based on the perforation of the base component.

41. The medical device assembly of claim 35, wherein the first portion and the second portion of the electrical component are configured to be destructively disconnected when the electrical component is in the second state.

42. The medical device assembly of claim 35, wherein the first portion and the second portion are configured to be separable independent of an additional mechanical component.

43. The medical device assembly of claim 35, wherein the electrical component is a first electrical component, and the medical device assembly further comprises:
a second electrical component electrically connected to the RFID circuit, wherein the second electrical component comprises an additional first portion and an additional second portion, and wherein the additional first portion of the second electrical component and the additional second portion of the second electrical component are configured to be separable; and
wherein the additional first portion is separated from the additional second portion, the RFID circuit is configured to provide a third signal, wherein the third signal is different from the first signal and the second signal.

44. The medical device assembly of claim 35, wherein the container comprises a single-use container.
